SEQUENCE LISTING (3 sequences)

SEQ ID NO: 1 - protein, 5 aa, Artificial Sequence
  synthetic peptide
GFGER

SEQ ID NO: 2 - protein, 6 aa, Artificial Sequence
  Artificial peptide
CGRGDS

SEQ ID NO: 3 - protein, 6 aa, Artificial Sequence
  Artificial peptide
  MISC_FEATURE (2)..(2): hydrophobic amino acid
  MISC_FEATURE (3)..(3): ornithine
GXXGER

(19) United States
(12) Patent Application Publication
(10) Pub. No.: US 2023/0285637 A1
(43) Pub. Date: Sep. 14, 2023

(54) MAGNETICALLY-ALIGNED SYNTHETIC EXTRACELLULAR MATRIX FIBERS WITHIN HYDROGEL

(71) Applicant: THE REGENTS OF THE UNIVERSITY OF MICHIGAN, Ann Arbor, MI (US)

(72) Inventors: Brendon M. BAKER, Ann Arbor, MI (US); Harrison L. HIRAKI, Honolulu, HI (US); Daniel L. MATERA, Albany, NY (US)

(73) Assignee: THE REGENTS OF THE UNIVERSITY OF MICHIGAN, Ann Arbor, MI (US)

(21) Appl. No.: 17/749,572

(22) Filed: May 20, 2022

Related U.S. Application Data
(60) Provisional application No. 63/191,253, filed on May 20, 2021.

(51) Int. Cl. A61L 27/44; A61L 27/22; A61L 27/52; A61L 27/48; A61L 27/20

(52) U.S. Cl. CPC A61L 27/446; A61L 27/22; A61L 27/52; A61L 27/48; A61L 27/20; A61L 2400/12; B82Y 5/00

(57) ABSTRACT

A composite material is provided. The composite material includes a hydrogel matrix having a three-dimensional geometry and fibers embedded and substantially uniformly distributed within the three-dimensional hydrogel matrix. The fibers have a substantially circular cross-sectional geometry and are anisotropically aligned. Methods of making and using the composite material are also provided.

### CROSS-REFERENCE TO RELATED APPLICATIONS

This application claims the benefit of U.S. Provisional Application No. 63/191,253, filed on May 20, 2021. The entire disclosure of the above application is incorporated herein by reference.

### REFERENCE TO A SEQUENCE LISTING

This application contains references to amino acid sequences and/or nucleic acid sequences which have been submitted concurrently herewith as the sequence listing text file entitled “Sequence Listing.TXT,” file size 892 bytes, created on May 20, 2021, as Appendix A. The aforementioned sequence listing is hereby incorporated by reference in its entirety pursuant to 37 C.F.R. § 1.52(e)(5).

### FIELD

The present disclosure relates to magnetically-aligned synthetic extracellular matrix (ECM) fibers within a hydrogel.

### BACKGROUND

This section provides background information related to the present disclosure which is not necessarily prior art.

Stromal ECM provides manifold biophysical cues that direct both physiologic and pathologic cell behavior. A major component of stromal ECM is fibrous proteins (e.g., collagens, fibronectin, and elastin) that serve as cell-adhesive scaffolding and provide structural and mechanical support to a variety of tissues. Cells dynamically deposit, reorganize, and respond to the fibrous architecture of the ECM. Through contact guidance cues, fibrous protein structures direct a variety of morphogenetic processes including tenogenesis, branching morphogenesis, and angiogenesis. Fibrous proteins are also heavily implicated in initiating and directing invasion from primary tumors during breast cancer progression. Second harmonic generation (SHG) imaging has provided valuable insights into collagen architecture during morphogenesis and disease progression. With this insight, biomaterials recapitulating aligned fibrous architectures have been developed to model and direct such processes in vitro.

Purified biopolymers, such as type I collagen and fibrin, have been used to model stromal ECM, as both possess fibrous topography. However, polymerization of these materials under typical conditions produces hydrogels with isotropic fibrous architecture due to the stochastic nature of fibrillogenesis. To better model highly anisotropic fibrous architecture, such as that found in tendons and around primary breast tumors, several approaches to align collagen fibers have been developed. Early methods to align collagen gels take advantage of the slight negative charge of collagen to align fibers with powerful magnetic fields. More recently, a diversity of methods to align collagen gels have emerged, including flow-induced alignment, embedding of magnetic particles which are dragged through the gel with an external magnetic field, application of tensile forces via stretching, and fibroblast-mediated reorganization of fibers. These methods create highly anisotropic collagen gels and have been instrumental in investigating how aligned fibrous architecture influences cell behavior. However, purified biopolymers typically have limited orthogonal control of relevant biophysical cues. For example, increasing type I collagen gel concentration leads to commensurate increases in fiber density, stiffness, and ligand density. In contrast, synthetic hydrogels (e.g., polyethylene glycol (PEG), methacrylated gelatin, and functionalized dextran) offer enhanced orthogonal tunability of these physical properties. However, these amorphous hydrogels typically lack fibrous architecture.

Electrospinning offers a means to generate fibrous topography that closely recapitulates the geometry and length-scale of fibrous proteins found in stromal ECM. The electrospinning process uses a voltage gradient to draw solid fibers from a charged polymer solution. Previous work with polyvinyl alcohol (PVA), poly(lactic-co-glycolic acid) (PLGA), and dextran methacrylate has shown that cell migration on electrospun, synthetic fiber matrices captures key aspects of cell migration in fibrous natural ECM proteins like type I collagen. Recent work has established means to generate suspended fiber segments within amorphous synthetic hydrogels. These hydrogel composites enable cell studies within topographically complex fibrous environments in which fiber density and stiffness can be orthogonally controlled. However, such composites rely on encapsulating fiber segments within a hydrogel, resulting in a random distribution of embedded fibers. Accordingly, anisotropically-aligned synthetic ECM fibers within a hydrogel are desired.

### SUMMARY

This section provides a general summary of the disclosure, and is not a comprehensive disclosure of its full scope or all of its features.

In various aspects, the current technology provides a composite material including a hydrogel matrix having a three-dimensional geometry and fibers embedded and substantially uniformly distributed within the hydrogel matrix, wherein the fibers have a substantially circular cross-sectional geometry and are anisotropically aligned.

In one aspect, the hydrogel matrix includes a polysaccharide, a polypeptide, a synthetic polymer, or combinations thereof.

In one aspect, the hydrogel matrix includes a polysaccharide selected from the group consisting of dextran, starch, cellulose, alginate, hyaluronic acid, chitosan, chitin, pectin, derivatives thereof, and combinations thereof.

In one aspect, the hydrogel matrix includes a polypeptide selected from the group consisting of collagen, fibronectin, gelatin, derivatives thereof, and combinations thereof.

In one aspect, the hydrogel matrix includes a synthetic polymer, the synthetic polymer being PEG.

In one aspect, the fibers include a polysaccharide, a synthetic polymer, or a combination thereof.

In one aspect, the fibers include a polysaccharide selected from the group consisting of dextran, starch, cellulose, alginate, hyaluronic acid, chitosan, chitin, pectin, derivatives thereof, and combinations thereof.

In one aspect, the fibers comprise a vinyl sulfone functionalized dextran.

In one aspect, the fibers include a synthetic polymer selected from the group consisting of polyvinyl alcohol (PVA), polyethylene oxide (PEO), poly(hydroxyethyl methacrylate) (PHEMA), polyvinylpyrrolidone (PVP), polyimide (PI), polyacrylate (PA), polyurethane (PU), a polyester, and combinations thereof.

In one aspect, the composite material further includes magnetic nanoparticles at least partially embedded within the fibers.

In one aspect, the magnetic nanoparticles are coated with a biomaterial.

In one aspect, the fibers are embedded within the hydrogel matrix at a fiber density of greater than or equal to about 1 v/v % to less than or equal to about 6 v/v %.

In one aspect, the hydrogel matrix is crosslinked with a peptide crosslinker.

In one aspect, the fibers are crosslinked with a peptide crosslinker.

In one aspect, cell adhesion promoters are coupled to at least one of the hydrogel matrix or the fibers.

In one aspect, the composite material further includes cells embedded within the hydrogel matrix.

In one aspect, the fibers have a fiber length of greater than or equal to about 100 μm to less than or equal to about 150 μm.

In various aspects, the current technology also provides a method of producing a composite material, the method including preparing a suspension of electrospun fibers having a substantially circular cross-sectional geometry, where at least a portion of the electrospun fibers have at least one magnetic nanoparticle at least partially embedded therein. The method comprises combining the suspension with a hydrogel precursor solution including polymer molecules to form a composite suspension; and crosslinking the polymer molecules within a magnetic field to form the composite material. The composite material formed from such a method thus includes the electrospun fibers embedded and substantially uniformly distributed within a three-dimensional hydrogel matrix formed from the polymer molecules and the electrospun fibers are anisotropically aligned.

In one aspect, the method further includes preparing the electrospun fibers by electrospinning a fiber mat having magnetic nanoparticles embedded within a plurality of continuous fibers; disposing a photomask over the fiber mat, the photomask including a plurality of apertures; applying ultraviolet (UV) light through the plurality of apertures to crosslink the continuous fibers at region exposed by way of the apertures and form the electrospun fibers; isolating the electrospun fibers from portions of the fiber mat that were blocked from being crosslinked by the photomask; and suspending the electrospun fibers in a solvent.

In one aspect, the apertures of the photomask have a diameter of greater than or equal to about 75 μm to less than or equal to about 250 μm and the electrospun fibers have a fiber length of greater than or equal to about 100 μm to less than or equal to about 150 μm.

In one aspect, the electrospinning is performed with a composition including a fiber precursor, a photoinitiator, and the magnetic nanoparticles at a density of greater than or equal to about 2.5 mg/mL to less than or equal to about 10 mg/mL.

In one aspect, the composite suspension is formed in, or transferred to, a sealed or water-tight container, and the method further includes periodically rotating the water-tight container about 180° to provide a substantially uniform distribution of the electrospun fibers within the composite suspension until the crosslinking is complete.

In one aspect, the method further includes generating the magnetic field between two magnets.

In one aspect, the magnetic field is characterized by a magnet flux density of greater than or equal to about 5 mT to less than or equal to about 1 T.

In one aspect, the method further includes adding a plurality of cells to the composite suspension.

In various aspects, the current technology additionally provides a method of preparing an implant that may be used for repairing a tissue having a damaged region in a subject in need thereof. The method may include growing cells of the tissue in a composite material until an artificial tissue is formed and implanting the composite material with the artificial tissue into the damaged region of the tissue. The composite material includes a hydrogel matrix having a three-dimensional geometry and fibers embedded and substantially uniformly distributed within the hydrogel matrix, wherein the fibers have a substantially circular cross-sectional geometry and a fiber length of greater than or equal to about 100 μm to less than or equal to about 150 μm and the fibers are anisotropically aligned.

In one aspect, the tissue is a tendon and the cells include tendon fibroblasts.

In one aspect, the tissue is a heart and the cells include cardiomyocytes.

In various aspects, the current technology further provides a method of modeling a cellular environment, the method including growing cells in a composite material until the cellular environment is formed, wherein the composite material includes a hydrogel matrix having a three-dimensional geometry and fibers embedded and substantially uniformly distributed within the hydrogel matrix, wherein the fibers have a substantially circular cross-sectional geometry and a fiber length of greater than or equal to about 100 μm to less than or equal to about 150 μm and the fibers are anisotropically aligned.

In one aspect, the cellular environment is breast tissue and the cells include breast tissue cells.

In one aspect, the cellular environment is vasculature and the cells include endothelial cells.

In one aspect, the cellular environment is cardiac tissue and the cells include cardiomyocytes, epicardial cells, cardiac fibroblasts, endothelial cells, endocardial cells, or combinations thereof.

In one aspect, the cellular environment is connective tissue and the cells include fibroblasts.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a flow chart showing a method of making a composite material in accordance with various aspects of the current technology.

FIG. 2 is an illustration of an electrospun fiber having magnetic nanoparticles embedded therein in accordance with various aspects of the current technology.

FIG. 3 is an illustration of a composite suspension prior to gelation in accordance with various aspects of the current technology.

FIG. 4 is an illustration of a composite material in accordance with various aspects of the current technology.

FIGS. 5A-5E show the fabrication and magnetic alignment of superparamagnetic iron oxide nanoparticle (SPION)-containing electrospun dextran vinyl sulfone (DVS) fiber segments. FIG. 5A is a schematic overview of DVS polymer electrospinning, collected fiber suspension within a bulk hydrogel precursor solution, and alignment of SPION-containing fibers within an externally applied magnetic field. FIG. 5B is a transmitted light image of SPIONs within electrospun DVS fibers with arrowheads indicating SPIONs. FIG. 5C shows top, front, and side views of a magnetic gelation chamber containing variably-spaced neodymium magnets to control magnetic field strength during hydrogel gelation. FIG. 5D is an AutoCAD rendering of the magnetic gelation chamber with an Arduino-controlled stepper motor to flip hydrogel composites during crosslinking and prevent fiber settling. FIG. 5E is an image of the final magnetic gelation chamber.

FIGS. 6A-6C show computational modeling of applied magnetic fields within a gelation chamber. FIG. 6A illustrates a model geometry with two cylindrical magnets within a spherical air area. The geometry has magnet orientation with North in the positive Z-direction. FIG. 6B is a graph showing quantified magnetic field strength in the Z-direction of the magnet axis over a range of magnet spacings. FIG. 6C is a visualization of magnetic flux density and field lines (arrows).

FIGS. 7A-7D show fiber alignment as a function of SPION density and magnet spacing. FIG. 7A show images of fiber alignment at 1 v/v % fiber density in three-dimensional DVS hydrogels across a range of encapsulated SPION densities at 6 cm magnet spacing. FIG. 7B is a FibrilTool quantification of anisotropic fiber alignment. FIG. 7C shows images of fiber alignment of 5 mg mL⁻¹ SPION fibers at 1 v/v % over a range of magnet spacings. FIG. 7D is a quantification of fiber alignment. All data are presented as mean±standard deviation (SD); * indicates a statistically significant comparison with p<0.05; A indicates significance against -Mag; # indicates significance against -SPION.

FIGS. 8A-8B show color map images based on fiber orientation for fibers aligned across SPION encapsulation densities and magnet distances.

FIGS. 9A-9G show that decreasing fiber length prevents entanglement at high fiber encapsulation density. FIG. 9A shows images of the alignment of full length fibers across a range of densities. Inserts show local regions of alignment (G) and entanglement (R). FIG. 9B is a graph showing anisotropy scoring across a range of fiber densities. FIG. 9C is a schematic of photomasking during photocrosslinking of fiber mats to define shorter fiber lengths. FIG. 9D is a quantification of fiber length as a function of photomask size. FIG. 9E shows images of the alignment of fiber segments produced with photomasks within three-dimensional hydrogel at 5 v/v % fiber density, and FIG. 9F is a graph of corresponding anisotropy scores. FIG. 9G is an image showing the cross-section of a 5 mm cylindrical hydrogel composite with fibers produced by a 150 μm photomask aligned by 6 cm magnet spacing. Insets show location regions of fibers aligned at boundaries perpendicular (R) and parallel (B) to fiber alignment and within the gel center (G). All data are presented as mean±SD; * indicates a statistically significant comparison with p<0.05.

FIGS. 10A-10E show that PVP-coated SPIONs improve cytocompatibility without compromising magnetic alignment. FIG. 10A shows images of Hoechst and propidium iodide staining of MCF10As with uncoated or PVP-coated SPIONs added to culture media for 12 hours. FIG. 10B is a quantification of MCF10A death as measured by % propidium iodide⁺ nuclei with either SPIONs directly added to media (SPION-treated) or SPIONs incubated in media and then removed prior to media transfer to cells (SPION-conditioned media). FIG. 10C shows images of Hoechst/propidium iodide staining of single MCF10As encapsulated alongside SPION fibers in DVS hydrogels after 12 hours of culture. Nonfibrous gel was exposed to a magnetic field (magnet). FIG. 10D is a corresponding quantification of % cell death. FIG. 10E is a graph showing the alignment of fibers containing SPIONs with or without PVP coating. All data are presented as mean±SD; * indicates a statistically significant comparison with p<0.05; A indicates significance against no SPION control.

FIGS. 11A-11D shows that fiber alignment directs the orientation and morphology of encapsulated tendon fibroblasts. FIG. 11A shows fluorescent images of primary mouse tendon fibroblasts (tenocytes) cultured in hydrogel composites for 7 days with grey arrowheads indicating stellate morphology cells and white arrowheads indicating uniaxially spread cells. FIG. 11B shows histograms of cell orientation as a function of fiber alignment. FIG. 11C is a full width-half max quantification of n=10 cell orientation distributions. FIG. 11D is an angular stratification of cell orientations as a function of fiber alignment. All data are presented as mean±SD; * indicates a statistically significant comparison with p<0.05.

FIGS. 12A-12G show that fiber alignment biases migration direction from multicellular MCF10A spheroids and induces cell-cell breakage events. FIG. 12A shows fluorescent images of cell outgrowth from multicellular MCF10A spheroids encapsulated in DVS hydrogel composites after 6 days. FIG. 12B shows higher magnification images including DVS fibers from location depicted by inset in FIG. 12A. FIG. 12C shows heatmap overlays created by an aggregate sum of binarized actin channels, and FIG. 12D shows rose plots of migratory cell nuclei location for n=25 spheroids per condition. FIGS. 12E-12F are quantifications of the total number of migratory cells and total migration distance stratified by outgrowth contiguity with the spheroid, respectively. FIG. 12G is a graph showing maximum invasion depth of individual outgrowths stratified by contiguity with the spheroid. All data are presented as mean±SD; * indicates a statistically significant comparison with p<0.05.

FIG. 13 shows images illustrating that migration from spheroids occurs predominantly as multicellular collective strands that contact guides along fiber segments biased in the direction of fiber alignment.

FIGS. 14A-14F show in vivo formation of a composite material comprising SPION-laden vinyl sulfone functionalized dextran (DexVS) fibers within hydrogels in a mouse in accordance with various aspects of the current technology. FIG. 14A shows intraoperative image from a tenotomy and hydrogel composite material implantation surgery. FIG. 14B shows a tendon/hydrogel construct composite material explanted 7 days post-operation. FIG. 14C shows a schematic and image of a magnetic device for aligning SPION-laden DexVS fibers within hydrogels. FIG. 14D shows confocal images of SPION fibers (left) and tendon progenitor cell (TPCs) (right) within composite hydrogels either gelled normally (top) or within the magnetic field device (bottom). FIG. 14E shows an image of isoflurane-sedated mouse with hind limb positioned between device magnets during tenotomy and gel implantation in accordance with certain aspects of the current technology. FIG. 14F shows a confocal image of resulting fibrous hydrogel composite localized to the wound gap with fibers aligned along the long axis of the transected tendon.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges. As referred to herein, ranges are, unless specified otherwise, inclusive of endpoints and include disclosure of all distinct values and further divided ranges within the entire range. Thus, for example, a range of “from A to B” or “from about A to about B” is inclusive of A and B.

Example embodiments will now be described more fully with reference to the accompanying drawings.

The current technology provides a composite material comprising a hydrogel that is three-dimensional and reinforced with fibers, i.e., the fibers are embedded within a three-dimensional hydrogel matrix. The fibers are uniformly or substantially uniformly distributed throughout the hydrogel matrix and are anisotropically aligned by way of embedded magnetic nanoparticles. By “substantially uniformly distributed,” it is meant that the distribution of the fibers may slightly vary from one portion of the hydrogel matrix to another by less than or equal to about 10 v/v %. The composite material mimics an ECM and is capable of directing morphogenetic processes, supporting mechanical loads, and facilitating cell migration. The current technology also provides methods of making and using the composite material.

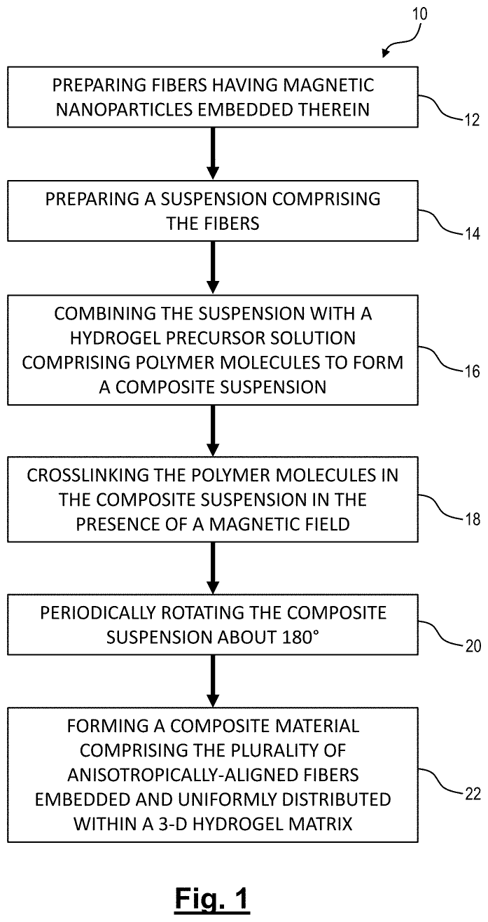

With reference to FIG. 1, the current technology provides a method 10 for producing a composite material. As shown in block 12, the method 10 comprises preparing fibers, wherein at least a portion of the fibers have at least one magnetic nanoparticle embedded therein. By “at least a portion,” it is meant that all or substantially all (i.e., greater than or equal to about 90%) of the fibers have at least one magnetic nanoparticle at least partially embedded therein.

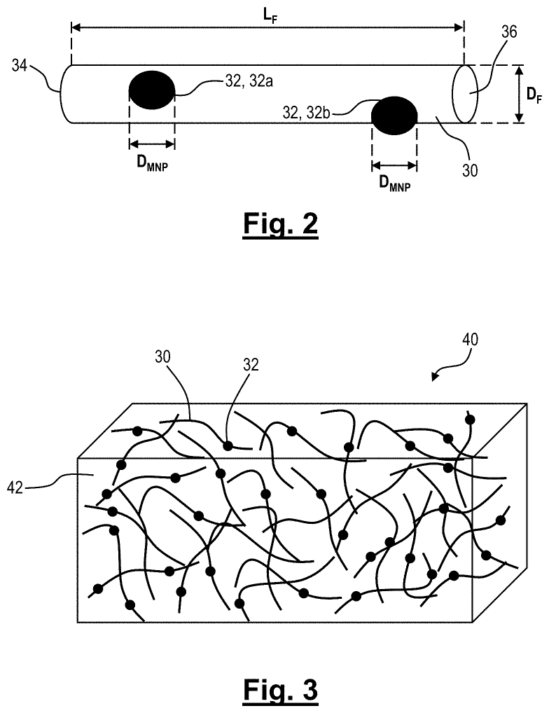

FIG. 2 provides an illustration of a fiber 30 prepared in in accordance with block 12 of the method 10. The fiber 30 comprises at least one magnetic nanoparticle 32, shown as a first magnetic nanoparticle 32a that is completely embedded within the fiber 30 and as a second magnetic nanoparticle 32b that is partially embedded with the fiber 30. Although the fiber 30 is depicted as having two embedded magnetic nanoparticles 32a, 32b, it is understood that fibers 30 of the current technology individually and independently have at least one magnetic nanoparticle 32 at least partially embedded therein and can include a plurality of magnetic nanoparticles 32.

The fiber 30 comprises a polymer, which may be a polysaccharide, a synthetic polymer, or a combination thereof. Non-limiting examples of polysaccharides include dextran, starch, cellulose, alginate, hyaluronic acid, chitosan, chitin, pectin, derivatives thereof, and combinations thereof. Non-limiting examples of synthetic polymers include PVA, PEO (including PEG in certain variations), PHEMA, PVP, PI, PA (e.g., polyacrylic acid (PAA)), PU, polyesters (including polycaprolactone (PCL), polylactic acid (PLA), and PLGA), derivatives thereof, and combinations thereof. The derivatives of the polysaccharides and synthetic polymers include base polysaccharides that are modified, e.g., coupled, with a functional group, a cell adhesion promoter (such as cell adhesion peptides), a light-emitting marker, or combinations thereof. Non-limiting examples of functional groups include vinyl sulfone, methacrylates, acrylates, diacrylates, norbornenes, maleimides, and combinations thereof. Non-limiting examples of cell adhesion promoters include peptides, such as arginylglycylaspartic acid (RGD peptide) and/or collagen integrin-binding peptides (e.g., having the amino acid sequence GFOGER (SEQ ID NO:1)); polypeptides (which may be full length polypeptides), such as collagen, fibronectin, laminin, and/or gelatin; derivatives thereof; and combinations thereof. Non-limiting examples of light-emitting markers include cyanine dyes, coumarins, rhodamines, xanthenes, quantum dots, and combinations thereof.

Each fiber 30 has an individual and independent fiber length L_{F}, when measured from a first end 34 to an opposing second end 36, optionally of greater than or equal to about 100 micrometers (μm) to less than or equal to about 150 μm, such as fiber lengths L_{F} of about 100 μm, about 105 μm, about 110 μm, about 115 μm, about 120 μm, about 125 μm, about 130 μm, about 135 μm, about 140 μm, about 145 μm, and about 150 μm. Each fiber 30 also has an individual and independent fiber diameter DF of greater than or equal to about 0.5 μm to less than or equal to about 5 μm, such as fiber diameters DF of about 0.5 μm, about 0.75 μm, about 1 μm, about 1.25 μm, about 1.5 μm, about 1.75 μm, about 2 μm, about 2.25 μm, about 2.5 μm, about 2.75 μm, about 3 μm, about 3.25 μm, about 3.5 μm, about 3.75 μm, about 4 μm, about 4.25 μm, about 4.5 μm, about 4.75 μm, and about 5 μm. In some aspects, the fiber length L_{F} approximates the size of a cell to be cultured in the composite material, where “approximates the size of a cell” means that the length L_{F} is within about 25% of the diameter of the cell. In other aspects, the fiber lengths L_{F} can be shorter than about 100 μm and/or longer than the about 150 μm. As shown in FIG. 2, the fiber 30 has a circular or substantially circular cross-sectional geometry, wherein “substantially circular” means that the cross-sectional geometry may not be a perfect circle and may have some deviations that define, for example, an oval or other imperfect circular shape. However, it is understood that alternative cross-sectional geometries can be employed.

The at least one magnetic nanoparticle 32 comprises Fe₂O₃, Fe₃O₄, or a combination thereof and may be a super paramagnetic iron oxide nanoparticle (SPION). However, it is understood that the magnetic nanoparticle material is not limiting and that the at least one magnetic nanoparticle 32 may include a compound that is not based on iron. The at least one magnetic nanoparticle 32 has a magnetic nanoparticle diameter D_{MNP} of greater than or equal to about 1 nm to less than or equal to about 250 nm or greater than or equal to about 1 nm to less than or equal to about 150 nm, such as magnetic nanoparticle diameters D_{MNP} of about 1 nm, about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, and about 250 nm.

Because the at least one magnetic nanoparticle 32 is at least partially embedded within the fiber 30, the cytotoxicity of the at least one magnetic nanoparticle 32 is less than the cytotoxicity of comparative magnetic nanoparticles that are not at least partially embedded within the fiber 30 or another material. Nonetheless, in some aspects, the at least one magnetic nanoparticle 32 is at least partially coated, including completely coated, with a biomaterial that further decreases cytotoxicity. Non-limiting examples of suitable biomaterials include PVP, PEG, PEI, PLGA, PLA, PCL, polypyrrole (PPy), poly(N-vinylpyrrolidine), polyanhydrides, poly(N-isopropylacrylamide) (NIPAAm), and combinations thereof.

With renewed reference to FIG. 1, the fibers (which are described above with reference to FIG. 2) are prepared, for example, by electrospinning. Although variations may exist, the electrospinning comprises applying an electric field between a droplet (e.g., a sessile droplet) of a polymer composition at the tip of a needle or pipette and a collector plate. The electric field causes a jet of liquid to issue from the droplet of polymer solution or melt to the collector plate. By rotating the collector plate, collected continuous fibers, which may be referred to as “fiber strings” or “fiber threads,” having lengths of greater than or equal to about 500 μm, greater than or equal to about 1 mm, or greater than or equal to about 1 cm can be formed into a fiber mat comprising the continuous fibers. Accordingly, the electrospinning results in the formation of a fiber mat defined by continuous fibers having the magnetic nanoparticles embedded therein. In an alternative method, the fibers can be prepared by mechanically drawing the fibers from a viscous polymer solution.

The polymer composition is a polymer solution or a polymer melt comprising a fiber precursor, the fiber precursor being the polysaccharide, the synthetic polymer, or the combination thereof, as discussed above with reference to FIG. 2, and a solvent. The solvent comprises water and an organic solvent, such as dimethylformamide (DMF) benzene, chlorobenzene, chloroform, cyclohexane, decalin, 1,2-dichloroethane, dimethyl sulfoxide, ethanol, methanol, 1,4-dioxane, ethyl acetate, ethylbenzene, hexane, methyl ethyl ketone (MEK), nitrobenzene, t-butyl acetate, tetralin, tetrahydrofuran (THF), toluene, and combinations thereof. The solvent comprises the water and the organic solvent at a water:organic solvent ratio of from about 1:100 to about 100:1, from about 1:10 to about 10:1, or from about 1:5 to about 5:1.

The polymer composition may also comprise the magnetic nanoparticles (optionally coated with the biomaterial) at a concentration of greater than or equal to about 1 mg/mL to less than or equal to about 15 mg/mL or greater than or equal to about 2.5 mg/mL to less than or equal to about 10 mg/mL, including concentrations of about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL, about 10 mg/mL, about 10.5 mg/mL, about 11 mg/mL, about 11.5 mg/mL, about 12 mg/mL, about 12.5 mg/mL, about 13 mg/mL, about 13.5 mg/mL, about 14 mg/mL, about 14.5 mg/mL, and about 15 mg/mL.

The polymer composition also comprises a photoinitiator. Non-limiting examples of suitable photoinitiators include lithium phenyl-2,4,6-trimethylbenzoylphosphinate (LAP), 2-hydroxy-1-[4-hydroxyethoxy)phenyl]-2-methyl-1-propanone (Irgacure 2959), benzophenone, 2,2-dimethoxy-2-phenylacetophenone, 2,2-dimethoxy-1,2-diphenylethan-1-one, 2-hydroxy-2-methyl-1-phenylpropanone, 1-hydroxy-cyclohexylphenylketone, isopropylthioxanthone, 2-ethylhexyl-(4-N,N-dimethyl amino)benzoate, ethyl-4-(dimethylamino)benzoate, and combinations thereof. The photoinitiator is included in the polymer composition at a concentration of greater than or equal to about 1 v/v % to less than or equal to about 20 v/v %.

The polymer composition can also comprise a visual marker, such as at least one of the light-emitting markers described herein. The visual marker may particularly be included when the polymer is not modified with a light-emitting marker. The visual marker is included in the polymer composition at a concentration of greater than or equal to about 1 v/v % to less than or equal to about 10 v/v %.

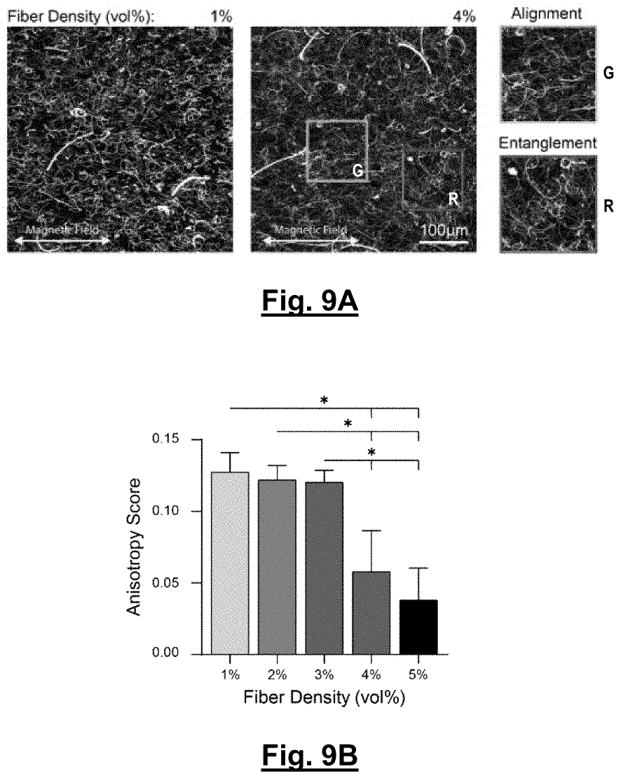

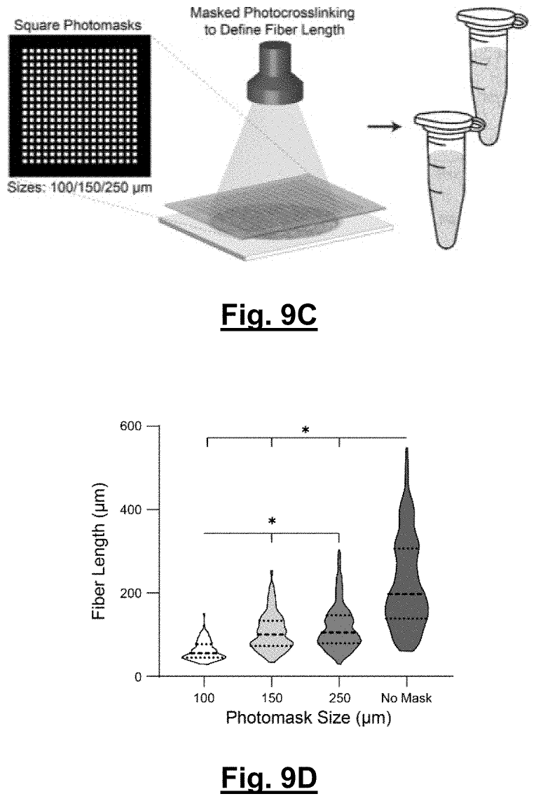

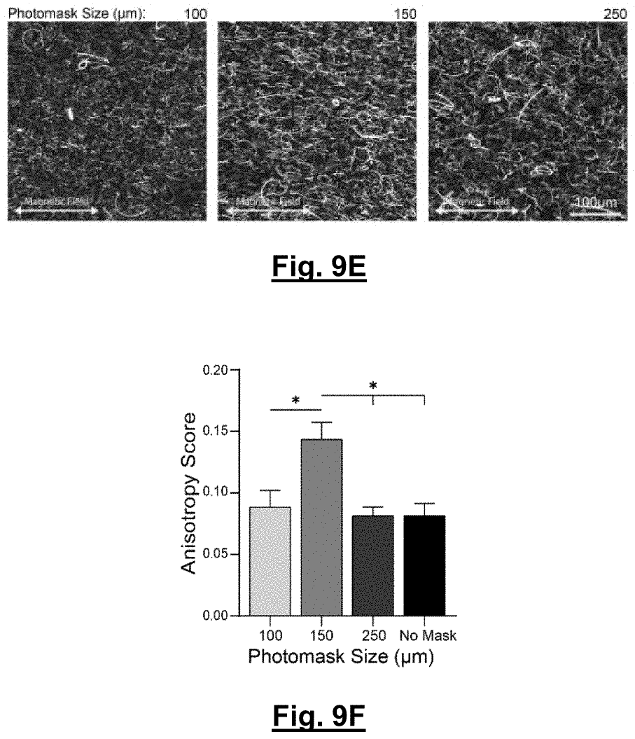

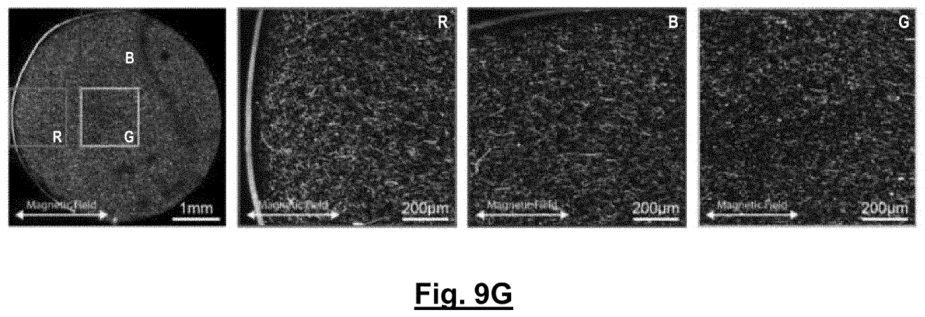

After the fiber mat is formed, the method of preparing the fibers comprises disposing a photomask over the fiber mat. The photomask is a UV light-blocking sheet or plate defining a plurality of apertures that are transparent to UV light. Each aperture of the plurality has a diameter of greater than or equal to about 25 μm to less than or equal to about 500 μm or greater than or equal to about 75 μm to less than or equal to about 250 μm, including diameters of about 25 μm, about 50 μm, about 75 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, and about 500 μm. The apertures can have a geometrical shape selected from the group consisting of a square, a rectangle, a circle, an oval, a triangle, a pentagon, a hexagon, and combinations thereof. However, it is understood that the geometrical shape of the apertures is not limited. An exemplary photomask is described in the below example with reference to FIG. 9C.

The method then comprises crosslinking portions of the fiber mat to form fiber segments, which are also referred to as “electrospun fibers” or “fibers,” by applying a UV light through the apertures of the photomask onto portions of the fiber mat that are exposed to the UV light by way of the apertures. Due to the presence of the photomask, only the portions of the fiber mat that are exposed to the UV light are crosslinked, resulting in the formation of a photopatterned, crosslinked fiber mat having segmented continuous fibers produced by the electrospinning. Accordingly, the method next comprises removing portions of the continuous fibers that were blocked from being crosslinked by way of the photomask to form the fibers (as described with reference to FIG. 2). The removing of the portions of the non-crosslinked continuous fibers is performed by transferring the photopatterned, crosslinked fiber mat into an aqueous solvent, such as water or phosphate buffered saline (PBS), as non-limiting examples, and successively centrifuging to generate a pellet of the fibers. Accordingly, the fibers are isolated from portions of the continuous fibers that were blocked from being crosslinked by the photomask.

In an alternative method, the continuous fibers are processed into the electrospun fibers by fracturing the continuous fibers using shear forces.

With continued reference to FIG. 1, the method 10 then comprises preparing a suspension comprising the fibers, as shown in block 14. The suspension is prepared by transferring the pellet to a fiber solvent, such as Michael-type addition buffer (MTAB; about 1 N NaOH, about 1 M HEPES, and about 1 mg mL⁻¹ phenol red in water), as a non-limiting example, at a concentration of greater than or equal to about 1 v/v % to less than or equal to about 20 v/v % or greater than or equal to about 5 v/v % to less than or equal to about 15 v/v %. In some aspects, the fibers are modified to include the cell adhesion promoters described herein by methods known in the art.

As shown in block 16, the method 10 then comprises combining the suspension with a hydrogel precursor solution to form a composite suspension. The hydrogel precursor solution comprises a hydrogel precursor, a crosslinking agent, and a hydrogel solvent. The hydrogel precursor comprises polymer molecules, such as polysaccharides, synthetic polymers, polypeptides, or combinations thereof. Non-limiting examples of polysaccharides include dextran, starch, cellulose, alginate, hyaluronic acid, chitosan, chitin, pectin, derivatives thereof, and combinations thereof. Non-limiting examples of synthetic polymers include PEO and PEG. However, in alternative aspects, the synthetic polymer can include PVA, PHEMA, PVA, PVP, PI, PA (e.g., PAA), PU, polyesters (including PCL, PLA, and PLGA), derivatives thereof, and combinations thereof, which can be cast into a polymer matrix (instead of a hydrogel matrix) using an organic solvent. Non-limiting examples of polypeptides include collagen, fibronectin, gelatin, derivatives thereof, and combinations thereof. The derivatives of the polysaccharides, synthetic polymers, and polypeptides include base polymers (polysaccharides, synthetic polymers, and polypeptides) that are modified with a functional group which facilitates crosslinking and hydrogel formation. Non-limiting examples of functional groups include vinyl sulfone, methacrylates, acrylates, diacrylates, norbornenes, maleimides, and combinations thereof. In some aspects, the hydrogel precursor is coupled to the cell adhesion promoter, as described above with references to the fibers. Accordingly, the cell adhesion promoter can be coupled to at least one of the fibers or the hydrogel precursor. Non-limiting examples of the crosslinking agent include peptide crosslinkers, e.g., VPMS crosslinker at a concentration of greater than or equal to about 2 mM to less than or equal to about 30 mM, dithiolated or diacrylated PEG chains, or dithiothreitol (DTT). The hydrogel precursor solution can also include at least one additive, such as an additional functional group, e.g., heparin binding peptide at a concentration of greater than or equal to about 750 μM to less than or equal to about 20 mM, as a non-limiting example. Non-limiting examples of the solvent include water, PBS, cell culture medium, and combinations thereof. The composite suspension is prepared in, or transferred to, a sealable (e.g., with a lid, cap, or the like) water-tight container that can be inverted without leaking.

The amount of the suspension added to the hydrogel precursor solution depends on the fiber concentration in the suspension and the level of fibers desired to be included in the composite material. Therefore, the fiber density of the composite material is tunable by adding a predetermined amount of the fiber suspension to the hydrogel precursor solution, such that a desired fiber density in the composite material is achieved. Alternatively, the fiber concentration of the fiber solution can be adjusted to a predetermined level so that a precalculated amount of the fiber solution can be added to the hydrogel precursor solution, such that a desired fiber density in the composite material is achieved. In some aspects, the fiber density in the composite suspension prepared by combining the suspension with the hydrogel precursor solution is greater than or equal to about 0.5 v/v % to less than or equal to about 10 v/v % or greater than or equal to about 1 v/v % to less than or equal to about 6 v/v %, including fiber densities of about 0.5 v/v %, about 1 v/v %, about 1.5 v/v %, about 2 v/v %, about 2.5 v/v %, about 3 v/v %, about 3.5 v/v %, about 4 v/v %, about 4.5 v/v %, about 5 v/v %, about 5.5 v/v %, about 6 v/v %, about 6.5 v/v %, about 7 v/v %, about 7.5 v/v %, about 8 v/v %, about 8.5 v/v %, about 9 v/v %, about 9.5 v/v %, and about 10 v/v %.

In some aspects, the composite suspension further comprises a plurality of cells to be cultured and grown in the composite material. The cells can be pluripotent, differentiated, or undifferentiated cells, such as cancer cells. Moreover, the cells can be primary cells obtained from a subject or immortalized cells, including immortalized cells of established cell lines that are known in the art. Exemplary pluripotent cells include stem cells, embryonic stem cells, induced pluripotent stem cells, mesenchymal stem cells, and epithelial stem cells. Differentiated cells include epidermal cells, endothelial cells, fibroblasts, tissue specific cells, and organ specific cells, such as cells normally found in a breast, lung, liver, heart (e.g., cardiomyocytes, epicardial cells, cardiac fibroblasts, endothelial cells, and endocardial cells), brain, pancreas, prostate, cervix, ovary, bladder, gall bladder, kidney, colon, stomach, oral cavity, skin, tendon (i.e., tenocytes, also referred to as tendon fibroblasts), cancer cells thereof, and combinations thereof, as non-limiting examples. The cells can be added to the composite material in at least one of the suspension or the hydrogel precursor.

In additional aspects, the composite suspension further comprises an adjunct agent, such as a growth factor, serum, antimicrobial (e.g., antibiotic, antiviral, antifungal), amino acid, and combinations thereof, as non-limiting examples.

An exemplary composite suspension 40 is shown in FIG. 3. The composite suspension 40 comprises a hydrogel precursor 42 and the fibers 30 having the at least one magnetic nanoparticle at least partially embedded therein. The fibers 30 are dispersed in the hydrogel precursor 42 in completely random orientations with no observable organization. Therefore, the distribution of the fibers 30 in the hydrogel precursor 42 is isotropic. If the composite suspension were to be crosslinked with no further processing, the resulting composite hydrogel would be characterized by this isotropic fiber orientation.

Referring back to FIG. 1, as shown in block 18, the method further comprises crosslinking the polymer molecules in the composite suspension in the presence of a magnetic field and, as shown in block 20, periodically rotating the composite suspension about 180° to prevent, inhibit, or minimize the fibers from settling and becoming localized at one portion of the composite suspension, which would result in a non-uniform distribution of the fibers in the composite suspension. By periodically rotating the composite suspension about 180° within the magnetic field, for example, every consecutive time interval of from about 5 seconds to about 60 seconds or from about 10 seconds to about 30 seconds until the crosslinking is complete, a uniform or substantially uniform distribution of the fibers within the composite suspension is maintained. The rotating is performed longitudinally so that the fibers flip end over end as the container is rotated by about 180°. In some alternative embodiments, the rotating is performed axially, such that the fibers remain in a constant longitudinal orientation between the magnets, but periodically rotate or spin about 180°, similar to an about 180° rotation of a wheel. It is understood that the composite suspension is rotated through the rotation of the sealed container in which the composite suspension is contained.

The magnetic field is generated by positioning a first magnet and a second magnet apart from each other. The first and second magnets magnetically communicate to form the magnetic field, which is characterized by a magnetic flux density of greater than or equal to about 1 mT to less than or equal to about 1 T, greater than or equal to about 1 mT to less than or equal to about 500 mT, or greater than or equal to about 5 mT to less than or equal to about 150 mT, such as about 1 mT, about 5 mT, about 10 mT, about 15 mT, about 20 mT, about 25 mT, about 30 mT, about 35 mT, about 40 mT, about 45 mT, about 50 mT, about 55 mT, about 60 mT, about 65 mT, about 70 mT, about 75 mT, about 80 mT, about 85 mT, about 90 mT, about 95 mT, about 100 mT, about 105 mT, about 110 mT, about 115 mT, about 120 mT, about 125 mT, about 130 mT, about 135 mT, about 140 mT, about 145 mT, about 150 mT, about 200 mT, about 250 mT, about 300 mT, about 350 mT, about 400 mT, about 450 mT, about 500 mT, about 550 mT, about 600 mT, about 650 mT, about 700 mT, about 750 mT, about 800 mT, about 850 mT, about 900 mT, about 950 mT, or about 1 T. A predetermined magnetic flux density can be achieved by adjusting the distance between the magnets. For example, the magnetic flux density increases as the magnets are brought closer together and decreases as the magnets are moved apart from each other. The strength of the magnet also may influence the distance provided between the magnets. As a non-limiting example, the magnets can be neodymium magnets.

The composite suspension is disposed between the first and second magnets in order to crosslink the polymer molecules in the presence of the magnetic field. When in the presence of the magnetic field, the magnetic nanoparticles embedded within the fibers are pulled in the direction of the magnetic field. As the magnetic nanoparticles are pulled under the magnetic field, they apply aligning forces to their corresponding fibers. As a result, the fibers align in the direction of the magnetic field. Therefore, the fibers transition from an isotropic orientation to an anisotropic orientation.

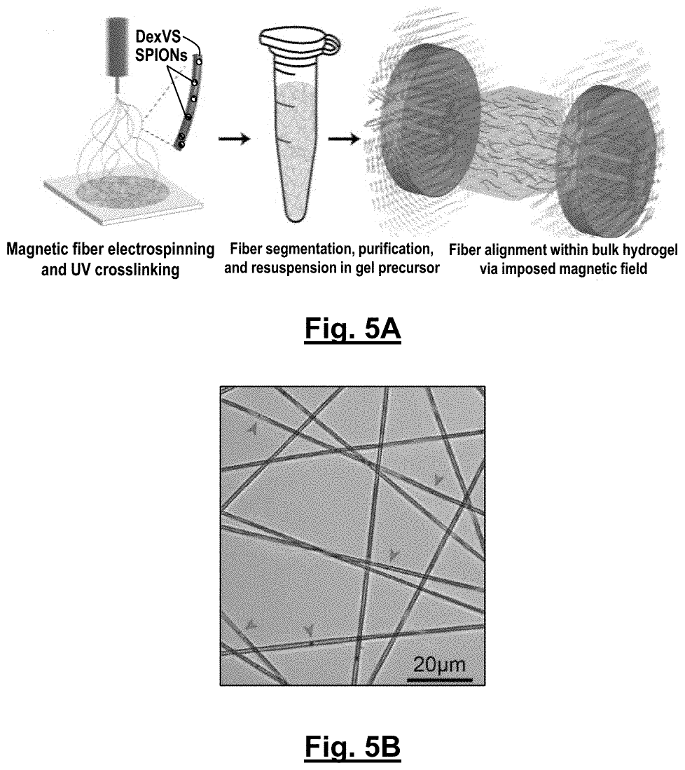

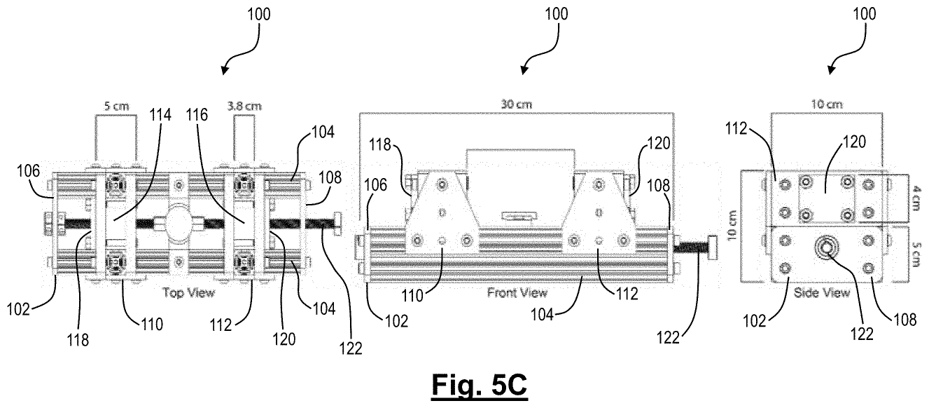

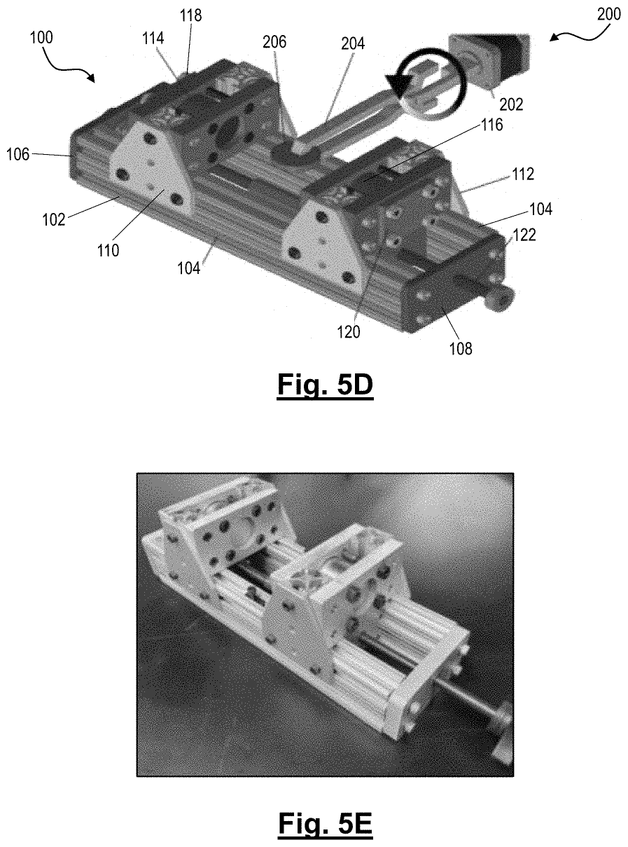

A system for applying the magnetic field and periodically rotating the composite suspension is described in the below example with reference to FIGS. 5C-5E. Briefly, the system includes a magnetic gelation chamber 100 comprising a base 102 having rails (e.g., tracks) 104 extending from a first end plate 106 to an opposing second end plate 108, a first carriage 110, and a second carriage 112, wherein the first and second carriages 110, 112 are operably coupled to, and movably engaged with, the rails 104. The first carriage 110 and the second carriages 112 carry a first magnet 114 and a second magnet 116, respectively. A first blocking plate 118 and a second blocking plate 120 are also disposed on outside surfaces of the first carriage 110 and the second carriage 112, respectively, that are not in between the magnets 114, 116. A crankshaft 122 is also operably coupled to the carriages 110, 112, such that when the crankshaft 122 is turned, the carriages 110, 112 slide on the rails 104 toward each other or away from each other, depending on the direction the crankshaft 122 is turned. As such, the distance between the magnets 114, 116 can be adjusted by rotating the crankshaft 122. It is understood that the lengths (and scale) shown in FIG. 5C are exemplary and non-limiting and can be increased or decreased. As shown in FIG. 5D, the system also includes a flipping apparatus 200 comprising a stepper motor 202 and a clamp 204, wherein the clamp 204 is operably coupled to the stepper motor 202. The clamp 204 is configured to hold a container 206 (e.g., a petri dish) containing the composite suspension. The stepper motor 202 is operable to periodically rotate the container 206 by way of the clamp 204.

Referring again to FIG. 1, the method 10 further comprises forming the composite material comprising the anisotropically-aligned fibers (also referred to as “magnetically-aligned fibers”) embedded and uniformly distributed within a three-dimensional hydrogel matrix, as shown in block 22. The hydrogel matrix, and thus the composite material, are formed when the crosslinking performed within the magnetic field is complete and gelation has occurred.

In some variations, the crosslinking of the polymer molecules in the composite suspension in the presence of the magnetic field (as shown in block 18) and the forming the composition material (as shown in block 22) are performed in vivo, and the method 10 does not include periodically rotating the composite suspension (as shown in block 20). In these variations, the magnetic field is provided by a magnetic resonance imaging (MRI) machine or other clinically available magnetic field generator.

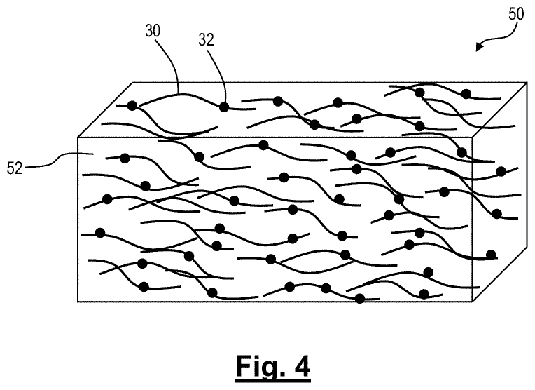

A composite material 50 formed from the method 10 is shown in FIG. 4. The composite material 50 comprises a hydrogel matrix 52 having a three-dimensional geometry and the fibers 30 embedded and uniformly or substantially uniformly distributed within the hydrogel matrix 52. By three-dimensional geometry, it is meant that the composite material has substantial dimensions in x, y, and z directions, for example, length, width, and height. As discussed herein, the fibers 30 have at least one magnetic nanoparticle 32 at least partially embedded therein. Moreover, as discussed above, the fibers 30 are anisotropically aligned. By anisotropically aligned, it is meant that the fibers 30 align, for example, longitudinally, such that physical properties of the composite material 50 are different when measured in a direction of the alignment versus when measured in a direction orthogonal to the direction of the alignment. As determined using the ImageJ plugin FibrilTool, the fibers 30 exhibit an anisotropy score of greater than or equal to about 0.05, greater than or equal to about 0.06, greater than or equal to about 0.07, greater than or equal to about 0.08, greater than or equal to about 0.09, greater than or equal to about 0.1, greater than or equal to about 0.11, greater than or equal to about 0.12, greater than or equal to about 0.13, greater than or equal to about 0.14, or greater than or equal to about 0.15, such as anisotropy scores of about 0.05, about 0.06, about 0.07, about 0.08, about 0.09, about 0.1, about 0.11, about 0.12, about 0.13, about 0.14, about 0.15, about 0.16, about 0.17, about 0.18, about 0.19, or about 0.2, where an anisotropy score of 0 indicates no order or alignment, i.e., a purely isotropic distribution, and an anisotropy score of 1 indicates a perfectly ordered and aligned distribution wherein all of the fibers 30 are perfectly parallel to each other, i.e., a purely anisotropic distribution. In some aspects, the cell adhesion promoter is coupled to at least one of the fibers 30 or the hydrogel matrix 52. Although not shown, the composite material 50 can include cells embedded therein that associate with, e.g., adhere to, the fibers 30. The composite material 50 mimics an ECM and is capable of directing morphogenetic processes, supporting mechanical loads, and facilitating cell migration.

The current technology also provides a method of preparing a tissue implant to repair a tissue having a damaged region or defect in a subject in need thereof, the subject being a human or non-human mammal, a fish, a bird, a reptile, or an amphibian. The method comprises growing cells of the tissue in the composite material described herein until an artificial tissue is formed. The method then comprises implanting the composite material including the artificial tissue into or on the damaged region of the tissue. The tissue is non-limiting and can be, for example, a tendon (wherein the cells comprise tendon fibroblasts), a heart (wherein the cells comprise cardiomyocytes), or a pancreas (wherein the cells comprise islet cells (alphas cells, beta cells, or a combination thereof), delta cells, pancreatic polypeptide cells (PP cells), or combinations thereof). In some aspects, the damaged region or defect is a hole or a tear that is congenial, a result of an accidental injury, or an aspect of a medical procedure or surgery. The implanting can be performed by creating an incision in the subject and manually disposing the composite material onto the damaged region of the tissue as a patch or by injecting the composite material onto the damaged region of the tissue by way of a syringe.

The current technology also provides a method of modeling a cellular environment, for example, for pathophysiologic studies. The method comprises growing cells in the composite material described herein until the cellular environment is formed. In some aspects, the cellular environment is normal breast tissue (and the cells comprises normal breast tissue cells), neoplastic tissue (and the cells comprise breast cancer cells), vasculature (and the cells comprise endothelial cells), cardiac tissue (and the cells comprise cardiomyocytes, epicardial cells, cardiac fibroblasts, endothelial cells, endocardial cells, or combinations thereof), or connective tissue (and the cells comprise fibroblasts). In certain aspects, the cellular environment is propagated until an artificial organ or organoid is formed.

The current technology yet further provides a method of repairing a tissue having a damaged region or defect in a subject in need thereof, the subject being a human or non-human mammal, a fish, a bird, a reptile, or an amphibian. The method comprises implanting the composite material into or on the damaged region of the tissue, wherein the composite material does not include cells prior to implantation, i.e., the composite material is cell-free. The tissue is non-limiting and can be, for example, a tendon or ligament or a portion of an organ, such as a heart, breast, lung, liver, brain, pancreas, prostate, cervix, ovary, bladder, gall bladder, kidney, colon, stomach, oral cavity, or skin, as non-limiting examples. In some aspects, the damaged region or defect is a hole or a tear that is congenial, a result of an accidental injury, or an aspect of a medical procedure or surgery. The implanting can be performed by surgically or endovascularly disposing the anisotropically-aligned composite material onto the damaged region of the tissue (e.g., as a patch). Alternatively, the implanting can be performed by injecting the hydrogel composite precursor solution onto or into the damaged region of the tissue as a gel or hydrogel via a syringe. Given the non-contact nature of magnetic alignment, the hydrogel composite precursor solution, e.g., the composite suspension, can be injected onto or into the damaged region and then aligned, for example, with the use of a MRI machine or any other clinically available magnetic field generator. In some aspects, cells from the subject's tissue adjacent to the damaged region or defect enter and infiltrate the patch or hydrogel composite precursor solution.

Embodiments of the present technology are further illustrated through the following non-limiting example.

### EXAMPLES

Fibrous ECM proteins provide mechanical structure and adhesive scaffolding to resident cells within stromal tissues. Aligned ECM fibers play an important role in directing morphogenetic processes, supporting mechanical loads, and facilitating cell migration. Various methods have been developed to align matrix fibers in purified biopolymer hydrogels, such as type I collagen, including flow-induced alignment, uniaxial tensile deformation, and magnetic particles. However, purified biopolymers have limited orthogonal tunability of biophysical cues including stiffness, fiber density, and fiber alignment. Here, synthetic, cell-adhesive fiber segments of the same length-scale as stromal fibrous proteins are generated through electrospinning. SPIONs embedded in synthetic fiber segments enable magnetic field-induced alignment of fibers within an amorphous bulk hydrogel. It is found that SPION density and magnetic field strength jointly influence fiber alignment and identify conditions to control the degree of alignment. Tuning fiber length allows the alignment of dense fibrous hydrogel composites without fiber entanglement or regional variation in the degree of alignment. Functionalization of fiber segments with cell adhesive peptides induces tendon fibroblasts to adopt a uniaxial morphology akin to within native tendon. Furthermore, the utility of this hydrogel composite to direct multicellular migration from MCF10A spheroids is demonstrated, and it is found that fiber alignment prompts invading multicellular strands to separate into disconnected single cells and multicellular clusters. These magnetic fiber segments can be readily incorporated into other natural and synthetic hydrogels and aligned with inexpensive and easily accessible rare earth magnets without the need for specialized equipment. Three-dimensional hydrogel composites where stiffness/crosslinking, fiber density, and fiber alignment can be orthogonally tuned provide insights into morphogenetic and pathogenic processes that involve matrix fiber alignment and enable systematic investigation of the individual contribution of each biophysical cue to cell behavior.

In this example, SPIONs are embedded within synthetic fiber segments during the electrospinning process to enable fiber alignment under an externally applied magnet field. Degree of fiber alignment within a three-dimensional amorphous hydrogel proves sensitive to SPION density as well as the strength of the imposed magnetic field. Computational modeling of the magnetic field reveals dependence upon magnet placement, where hydrogels appropriately positioned during crosslinking can achieve homogeneous alignment of constituent fibers. It is found that fiber length influences the frequency of fiber entanglement as a function of fiber density during magnetic alignment. Finally, the use of magnetic fiber alignment within the hydrogel composite system to control encapsulated tendon fibroblast (tenocyte) alignment and elicit directional migration of a breast epithelial cell line is demonstrated. Interestingly, it is found that fiber alignment not only biases epithelial cell migration direction, but also promotes cell-cell breakage events leading to a switch in three-dimensional migration phenotype.

### Materials and Methods

Reagents: All reagents are purchased from Sigma Aldrich and used as received, unless otherwise stated.

Synthesis of modified DVS: Dextran is functionalized with vinyl sulfone pendant groups using a previously described protocol. Briefly, linear high molecular weight dextran (MW 86,000 Da; MP Biomedicals) is reacted with pure divinyl sulfone (Fisher) under basic conditions (pH 13.0). Functionalization is terminated through pH adjustment to 5.0 with hydrochloric acid. Reaction products are dialyzed against milli-Q water for 3 days, with water changed twice daily. Purified products are then lyophilized for 3 days and reconstituted at 100 mg mL⁻¹ in a Michael-type addition buffer (MTAB; 1 N NaOH, 1 M HEPES, 1 mg mL⁻¹ phenol red in milli-Q water).

DVS fiber segment fabrication: DVS is dissolved at 0.6 g ml⁻¹ in a 1:1 mixture of milli-Q water and DMF. SPIONs with or without PVP coating (US Research Nanomaterials) are added at 2.5, 5, or 10 w/v %. LAP photoinitiator (10 v/v %), and methacrylated rhodamine (2.5 v/v %) (Polysciences, Inc.) are added to the solution to facilitate photoinitiated crosslinking and fluorescent visualization, respectively. Polymer solutions are electrospun in a humidity-controlled glove box held at 21° C. and 30-35% relative humidity. Electrospinning is performed at 0.25 ml hr⁻¹ flow rate, 7 cm gap distance, and −9.0 kV voltage onto a grounded copper collective surface. Fibers are collected on glass cover slides and crosslinked under ultraviolet light (100 mW cm⁻²) for 20 seconds. Custom-fabricated chrome photomasks are placed over fiber mats during UV photocrosslinking to control fiber length. Fiber mats are detached from cover slides into milli-Q water and broken into individual fiber segments. Fiber segments are purified through a series of centrifugation steps to remove uncrosslinked polymer and entangled fibers before resuspension in MTAB at 10 v/v %. Prior to encapsulation within bulk hydrogels, fibers are coupled with 2.0 mM RGD (CGRGDS (SEQ ID NO:2); CPC Scientific) via Michael-type addition to enable eventual cell adhesion.

Magnetic gelation chamber fabrication: The magnet housing apparatus is created with solid ½″ aluminum and 1.00″×2.00″ T-slotted aluminum. Two N52 neodymium magnets (K&J Magnetics) are housed in carriages made from solid aluminum, securing the magnets on either side with a hole removed at the face of the magnet. The carriages are attached to the T-slotted aluminum rails on the base of the setup, which aligns the carriages and allows them to controllably slide along the rails with a crankshaft. A three-dimensional printed clamp is designed to hold a petri dish containing fiber-reinforced hydrogel composites within the center of the magnet axis. To prevent encapsulated fibers and cells from settling during hydrogel crosslinking, an Arduino-controlled stepper motor with a three-dimensional-printed grip is programmed to flip the petri dish 180° every 20 seconds.

Computation visualization of magnet field lines: COMSOL Multiphysics software is used to quantify magnetic flux densities and visualize field lines between magnets. A three-dimensional stationary study modeling magnetic fields generated without current (permanent magnet) is performed. Two cylinders (1.905×3.81 cm) modeling the two magnets are placed within a 15-25 cm radius sphere to model air impedance. Surface flux density of the two cylinders is set to 661.9 mT, the innate surface field of N52 neodymium magnets. A single slice in the X-Z plane is generated to visualize magnetic flux density around and between the magnets. White arrows are overlaid to visualize magnetic field lines with the arrow size logarithmically proportional to strength of magnet flux along the field line. To generate one-dimensional plots of magnetic flux density in the Z-direction across various magnet spacings, flux density values are extracted from a path along the magnet axis.

Hydrogel formation and fiber alignment: DVS gels are formed via an analogous click reaction at 3.5 w/v % with 9.64 mM VPMS crosslinker and heparin binding peptide (2 mM). All hydrogel precursor solutions are made in PBS. To create fibrous hydrogels, a defined stock solution (10 v/v %) of suspended fiber segments in MTAB is mixed into hydrogel precursor solutions prior to gelation. Via controlling the dilution of the fiber suspension, fiber density is tuned at a constant hydrogel weight percentage and bulk stiffness. Hydrogel precursor solutions are injected into 5 mm in diameter polydimethylsiloxane gaskets and crosslinked at 37° C. for 1 hour. To align encapsulated fibers, hydrogels are crosslinked between the two magnets of magnetic gelation chamber.

Cell lines and culture: Mouse resident tenocytes were harvested via primary tenocyte harvesting with a previously established protocol. Briefly, mouse tail tendons are encapsulated in 2 mg ml⁻¹ collagen I (Advanced Biomatrix), allowing cells to proliferate into the gel for 11 days. Gels are then digested with 0.25 mg ml⁻¹ collagenase from C. histolyticum, and cells are centrifuged out. Tenocytes are cultured in DMEM supplemented with 10 v/v % fetal bovine serum (Fisher) and 1 v/v % penicillin/streptomycin/amphotericin B. Tenocytes are passaged near confluency at a 1:2 ratio and used for studies until passage 3. For three-dimensional hydrogel encapsulation studies, media is additionally supplemented with 50 ng ml⁻¹ L-ascorbic acid-2-phosphate and transforming growth factor-β3 (Peprotech). Human mammary epithelial cells MCF10A (ATCC) are cultured in DMEM/F12 (1:1) supplemented with 5 v/v % horse serum (Fisher), 20 ng mL⁻¹ rhEGF (Peprotech), 0.5 mg mL⁻¹ hydrocortisone, 100 ng mL⁻¹ cholera toxin, and 10 μg mL⁻¹ insulin (Fisher). MCF10As are passaged at confluency at a 1:4 ratio and used for studies until passage 8. Then, MCF10As are detached with 0.25% trypsin-EDTA (Life Technologies), counted, and formed into 200 cell-sized spheroids overnight in inverse pyramidal PDMS microwells (AggreWell™, Stem Cell Technologies) treated with 0.5% Pluronic F-127 to prevent cell adhesion. All cells are cultured at 37° C. and 5% CO₂.

Cytotoxicity screens: SPIONs with or without PVP coating are suspended in complete MCF10A media over a range of densities. To create SPION conditioned media, SPIONs are incubated in complete MCF10A media for 48 hours and then centrifuged out at 20,000 rcf for 30 minutes. To conduct a two-dimensional monolayer assay, MCF10A cells plated on glass coverslips are exposed to SPION containing media or SPION conditioned media for 12 hours, then incubated in serum free MCF10A media with Hoechst stain (1 μg/ml) and propidium iodide (1 μg/ml) for 20 minutes prior to fixing. To conduct a three-dimensional hydrogel assay, DVS fiber segments containing SPIONs with or without PVP coating are coencapsulated with single MCF10A cells (1000000 cells mL⁻¹) in DVS hydrogels. After 12 hours in culture, hydrogels are incubated in serum free MCF10A media with Hoechst stain (2 μg/ml) and propidium iodide (2 μg/ml) and incubated on a rocker plate at 0.33 Hz for 1 hour to enhance diffusive transport prior to fixing.

Single cell spreading studies: Primary-derived tenocytes (5000000 cells mL⁻¹) and fiber segments (3 v/v %) are coencapsulated in DVS hydrogels. Studies are maintained in complete tenocyte media for 7 days, with media replenished every other day.

Spheroid migration studies: MCF10A spheroids are harvested and centrifuged to remove residual single cells. Spheroids (6000 per mL of gel) and fiber segments (3 v/v %) are simultaneously encapsulated in DVS hydrogels. Studies are cultured in complete MCF10A media for 6 days, with media replenished every other day.

Fluorescence, staining, and microscopy: Samples are fixed with 4% paraformaldehyde for 1 hour at room temperature. To visualize the actin cytoskeleton and nuclei, samples are stained with phalloidin and DAPI for 1 hour at room temperature. For immunostaining, gels are additionally permeabilized in PBS containing Triton X-100 (5 v/v %), sucrose (10 w/v %), and magnesium chloride (0.6 w/v %) and blocked in 4% BSA. Fluorescent imaging is performed with a Zeiss LSM 800 laser scanning confocal microscope. For migration analysis, Z-stacks are acquired with a 10× objective. High-resolution images are acquired with a 40× objective. All images are presented as maximum intensity projections.

Cell migration analysis: A previously established custom MATLAB image analysis code is used to extract morphometric data from spheroid migration studies. Briefly, max intensity projections of spheroid nuclei and F-actin channels are separately thresholded and object size filtered to remove background. A user-drawn ellipsoidal ROI covering the spheroid body is used to separate the spheroid body from migratory cells within outgrowths. The code segments F-actin structures into individual outgrowths, which are defined as either contiguous or noncontiguous based on contiguity with the spheroid body. A separate function segments overlapping nuclei to identify all nuclei within outgrowths. Individual outgrowth F-actin masks are used to determine migration distance into the surrounding hydrogel utilizing a separate custom function. Corresponding individual nuclei masks are used to determine nuclei locations, nuclear counts, and mark noncontiguous outgrowths as either multicellular clusters or single cells. All individual outgrowth nuclei and F-actin masks are then summed to produce final images of nuclei and F-actin channels. Individual outgrowth nuclei and F-actin masks are saved with counted nuclei or plotted lengths, respectively, and assigned an index to address discrepancies or outliers within final quantified data. Resulting data are stratified by contiguity with the spheroid body and exported to a spreadsheet containing individual outgrowth indices, number of migratory cells, outgrowth areas, and migration distances. Finally, spheroid body and outgrowth masks are summed across all analyzed spheroids to produce heatmap overlays.

Statistics: Statistical significance is determined by one-way analysis of variance (ANOVA) with post-hoc analysis (Tukey test), with significance indicated by p<0.05. All data are presented as mean±SD.

### Results

Fabrication of Magnetically Responsive Electrospun Fiber Segments.

To create fiber segments on the same length-scale as fibrous proteins found in stromal ECM, DVS polymer solution is electrospun to produce fibers approximately 2 μm in diameter. Electrospun fiber mats are processed into suspensions of fiber segments, which can then be encapsulated in three-dimensional hydrogels and aligned by an externally applied magnetic field, as shown in FIG. 5A. SPIONs added to the electrospinning solution are stably encapsulated within fibers upon photocrosslinking (see arrowheads in FIG. 5B). To define the strength of an imposed magnetic field during hydrogel gelation, the magnetic gelation chamber 100 shown in FIG. 5C is designed with adjustable spacing of two N52 neodymium permanent magnets 114, 116. The setup includes an aluminum base 102 and rails 104, upon which two magnet carriages 110, 112 housing the neodymium magnets 114, 116 can be controllably spaced with the crankshaft 122 over a range of 6-20 cm. Referring to FIG. 5D, a hydrogel precursor solution containing DVS fiber segments is crosslinked within a petri dish 206 positioned between the two magnets 114, 116. To prevent fibers or cells from settling during hydrogel crosslinking, the clamp 204 attached to an Arduino-controlled stepper motor 202 flips the petri dish 206 180° within the magnetic field every 20 seconds during gelation. This setup enables facile control over fiber alignment via magnet spacing and resulting magnetic field strengths, as shown in FIG. 5E.

Computational Visualization of Magnetic Field Lines and Field Strength.

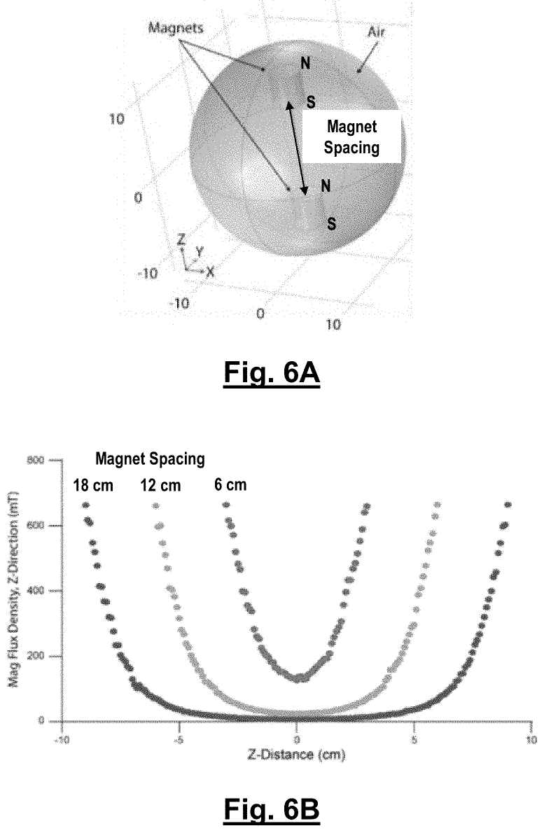

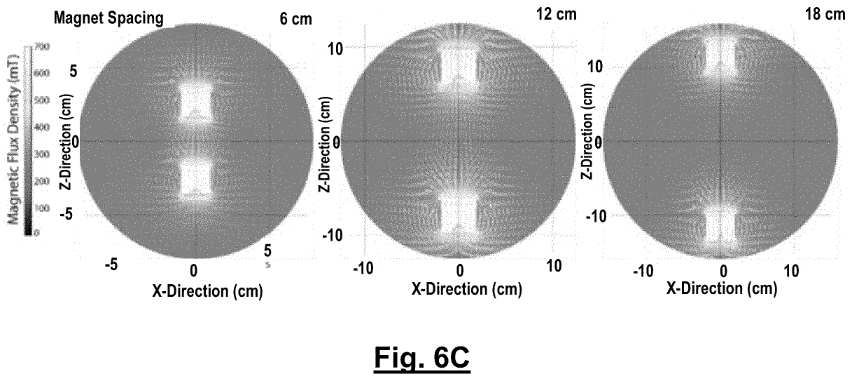

Magnetic field lines produced by a single permanent magnet resemble concentric ellipses radiating from the magnet's north to south pole. When opposite poles of two juxtaposed permanent magnets are aligned, field lines combine and densify as a function of spacing between the magnets. To determine the strength of the magnetic field produced within the magnetic gelation chamber, magnetic flux density is modeled using COMSOL. A three-dimensional model of two permanent magnets is created by placing two cylinders of equivalent geometry within a sphere to model air impedance, as shown in FIG. 6A. Surface fields of 669.1 mT are set at the cylinder surfaces to model N52 neodymium boundary conditions. Magnet flux density along the major magnet axis (Z-axis) is determined across a range of magnet spacings to quantitate the magnetic field strength applied to centrally positioned hydrogels, as shown in FIG. 6B. Flux density along the Z-axis is parabolic in strength—highest at the magnet surfaces and decaying exponentially to the center position between magnets (Z=0 cm). The smallest magnet spacing achievable (6 cm) produces a flux density of 126.9 mT at the center. Increasing magnet spacing to 12 and 18 cm significantly decreases flux density to 24.8 and 7.5 mT, respectively. To better visualize field lines between magnets, flux density heat maps are generated and overlaid with white arrows logarithmically proportional to regional flux densities, as shown in FIG. 6C. Field lines are parallel to magnet axis orientations and decayed exponentially once outside of the radius the magnets (X<−1.905 cm or X>1.905 cm). Thus, hydrogel composites positioned within the central region of the magnets are exposed to a nearly homogeneous magnetic field with field lines running parallel to the magnet axis.

Degree of Fiber Alignment is Jointly Regulated by SPION Density and Magnet Spacing.

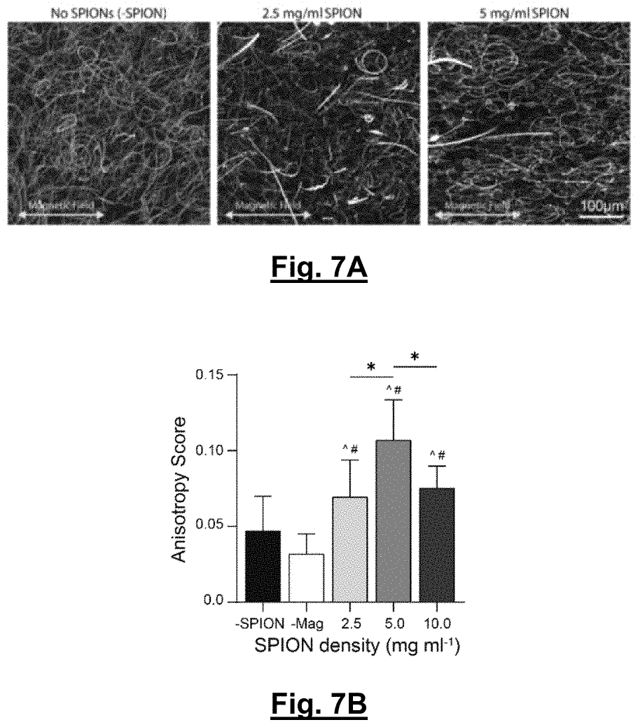

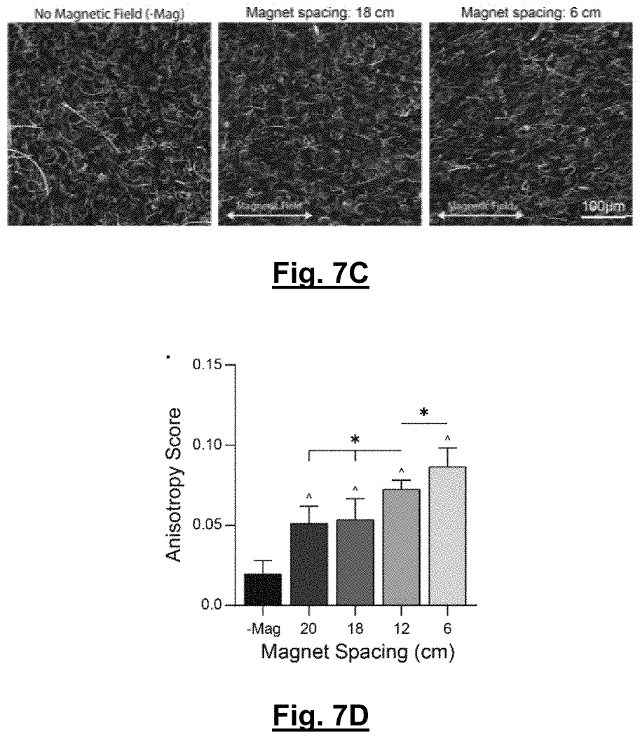

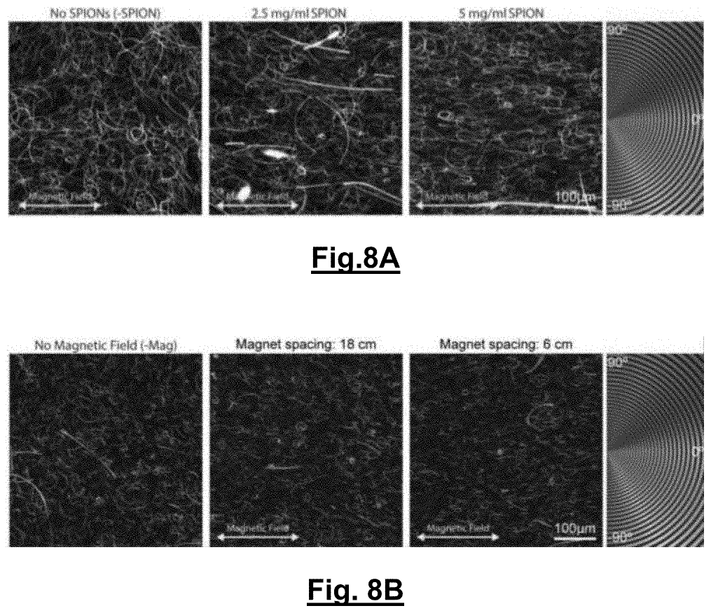

To optimize DVS fiber alignment, the density at which SPIONs are incorporated into the DVS electrospinning solution is first modulated. A slight decrease in fiber segment yield occurs with increasing SPION density (data not shown), likely due to the SPIONs interfering with the electrospinning process. Fiber segments are encapsulated in three-dimensional DVS gels at 1 v/v % and aligned at a magnet spacing of 6 cm, as shown in FIG. 7A. Degree of fiber alignment is quantified via anisotropy score generated with the ImageJ plugin FibrilTool. Hydrogels containing fibers without SPIONs crosslinked at 6 cm magnet spacing result in randomly oriented fibers (FIG. 7B; -SPION), indicating DVS fiber segments are not innately responsive to a magnetic field. Hydrogels containing fibers with the highest SPION density (10 mg mL⁻¹) crosslinked outside of the magnetic gelation chamber also result in randomly oriented fibers (FIG. 7B; -Mag), indicating SPION-containing fibers do not align in the absence of an external magnetic field. In contrast, hydrogels containing SPION fibers crosslinked within the magnetic field contain aligned fibers oriented in the direction of the magnetic field. The highest degree of fiber alignment results from a SPION density of 5 mg mL⁻¹, suggesting a density of 2.5 mg mL⁻¹ is below an optimal density required for magnetic forces to align fibers. Conversely, at 10 mg mL⁻¹, SPIONs begin to form large aggregates within the electrospinning solution, decreasing the total amount retained in fiber segments and therefore limiting alignment. Next, alignment of 5 mg mL⁻¹ SPION-containing fibers is assessed across a range of magnetic field strengths by varying the spacing between the two magnets, as shown in FIG. 7C. A step-wise decrease in fiber alignment is observed with increasing magnet spacing (see FIG. 7D), indicating fiber segments can be aligned with field strengths between 5-125 mT (see FIGS. 6A-6C) and that the degree of alignment is a function of both SPION density and field strength. To further visualize degree of fiber alignment, OrientationJ is utilized to produce color map images based on fiber orientation for fibers aligned across SPION encapsulation densities and magnet distances, as shown in FIGS. 8A-8B.

Decreasing Fiber Length Prevents Entanglement at High Fiber Density.

Previous reports on approaches to align type I collagen gels have noted collagen fiber entanglement. As a high fiber density is key to modeling fibrous tissues, such as tendons and the stroma of breast tissue, during cancer progression, it is next determined whether increases in fiber density lead to entanglement. Fiber density is modulated through the input fiber volume fraction of the hydrogel precursor solution over a range of 1-5 v/v % and gels are crosslinked at a magnet spacing of 6 cm. At fiber densities at or below 3 v/v %, highly anisotropic fiber alignment is achieved with minimal evidence of entanglement, as shown in FIGS. 9A-9B. However, at 4 v/v % fiber density, entanglement is apparent, which leads to a significant decrease in fiber alignment, as shown in FIG. 9B. Within these gels, heterogeneously distributed regions of localized fiber alignment versus entanglement are observed (FIG. 9A, third and first insets). At 5 v/v % fiber density, nearly all fibers are entangled in large clumps, leading to an anisotropy score similar to nonaligned fibers (FIGS. 7A-7D). As fiber entanglement occurs in regions where long fiber segments are coencapsulated in high proximity, alignment is attempted to be maintained at higher fiber densities by decreasing fiber segment length. To do so, chrome photomasks are placed over electrospun fiber mats during photocrosslinking, as shown in FIG. 9C. Photomasks with arrays of square patterns (100, 150, or 250 μm) yield fiber segments spanning 60-120 μm in average length; in contrast, fibers generated without photomasking are on average 225 μm in length, with considerably larger variance. To test if shorter fibers diminish entanglement despite high encapsulation density, 5 v/v % fibrous hydrogels are crosslinked at 6 cm magnet spacing, as shown in FIG. 9D. Fibers created with the 100 and 150 μm photomasks are highly aligned and show little evidence of entanglement, while gels containing 250 μm photomasked fibers possess regions of entanglement similar to fibers generated without photomasking, as shown in FIG. 9E. Despite the lack of evident entanglement in either hydrogel, gels containing 150 μm photomasked fibers have a significantly higher anisotropy score compared to gels containing 100 μm photomasked fibers. This difference is likely due to the influence of object length in FibrilTool's calculation of anisotropy score, as shown in FIG. 9F. To determine if rigid boundaries locally influence fiber alignment, a cross-section of a 5 mm cylindrical hydrogel composite containing 150 μm photomasked fibers is imaged. As shown in FIG. 9G, no regional differences in fiber alignment at gel boundaries perpendicular or parallel to fiber alignment are observed, indicating that magnetic alignment overcame any flow-induced alignment along boundaries. In sum, magnetic alignment of 5 mg mL⁻¹ SPION-containing fibers optimally sized by photomasking results in homogeneous alignment.

SPION Encapsulation within Fiber Segments Prevents Cytotoxic Interaction with Cells.

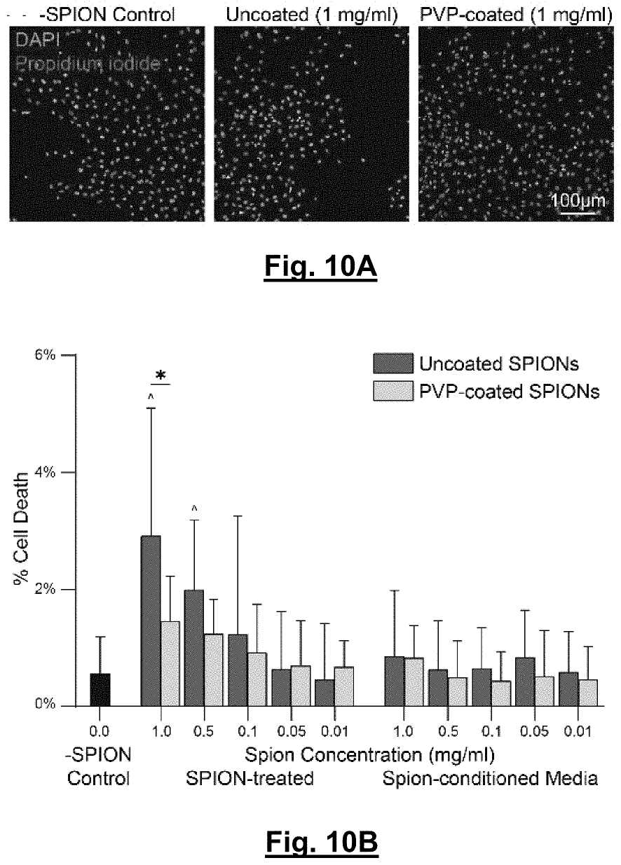

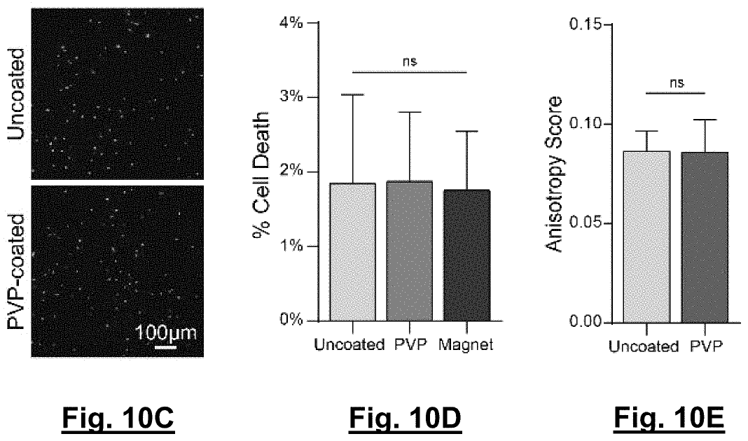

The presence of charged SPIONs has previously been reported to be cytotoxic. To determine if the SPIONs used here are cytotoxic and to test if cytotoxicity results from direct interaction of SPIONs with cells versus changes in media ion concentrations due to the addition of SPIONs, SPIONs or SPION-conditioned media is added to MCF10A mammary epithelial cell monolayers. Cell death, assessed via staining with membrane-impermeable propidium iodide, is SPION dose-dependent with 1 and 0.5 mg mL⁻¹ SPION concentration in media resulting in significant increases in cell death relative to controls, as shown in FIGS. 10A-10B. SPION-conditioned media at any concentration tested did not increase cell death above control levels, suggesting cytotoxicity results from direct cell interactions with SPIONs rather than changes in media ion concentrations. PVP coating of biomaterials has previously been reported to reduce cytotoxicity. As such, cytotoxicity experiments with PVP-coated SPIONs were repeated. A PVP-coated SPION dose-dependent increase in cell death is again observed, but cell death upon addition of 1 mg mL⁻¹ SPIONs is lower with PVP coating. PVP-coated SPION-conditioned media did not induce cell death above control levels, as shown in FIG. 10B. The significant decrease in cell death at the highest SPION concentration indicates PVP coating decreases cytotoxicity. However, it is worth noting that the degree of cell death is minimal (less than 3%) regardless of PVP coating. Next, to assess cytotoxicity when SPIONs are embedded within fiber segments, single MCF10A cells and fibers containing 5 mg mL⁻¹ of SPIONs with or without PVP coating are coencapsulated, as shown in FIG. 10C. After 12 hours in culture, no difference in cell death is observed, indicating insignificant SPION escape from fiber segments and limited cytotoxicity. Furthermore, as shown in FIG. 10D, no increase in cell death is observed in nonfibrous gels exposed to the strongest magnetic field (6 cm magnet spacing), indicating cells are not negatively affected by an externally applied magnetic field. As shown in FIG. 10E, the ability to electrospin SPION-containing fibers or align fibers within three-dimensional gels is not altered by PVP coating, and therefore PVP-coated SPIONs are used in all subsequent studies.

Fiber Alignment Directs Uniaxial Spreading in Primary-Derived Mouse Tenocytes.

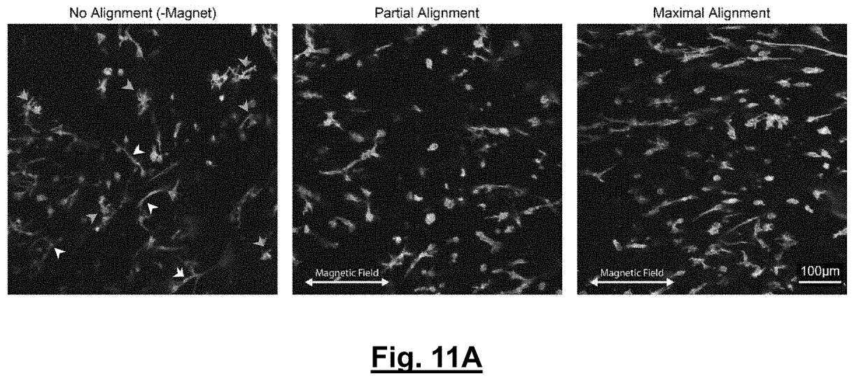

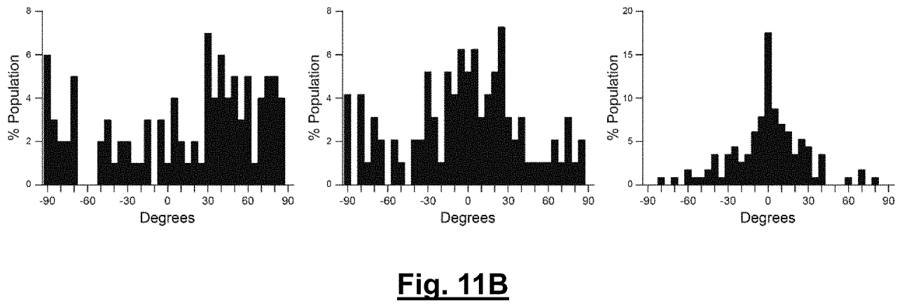

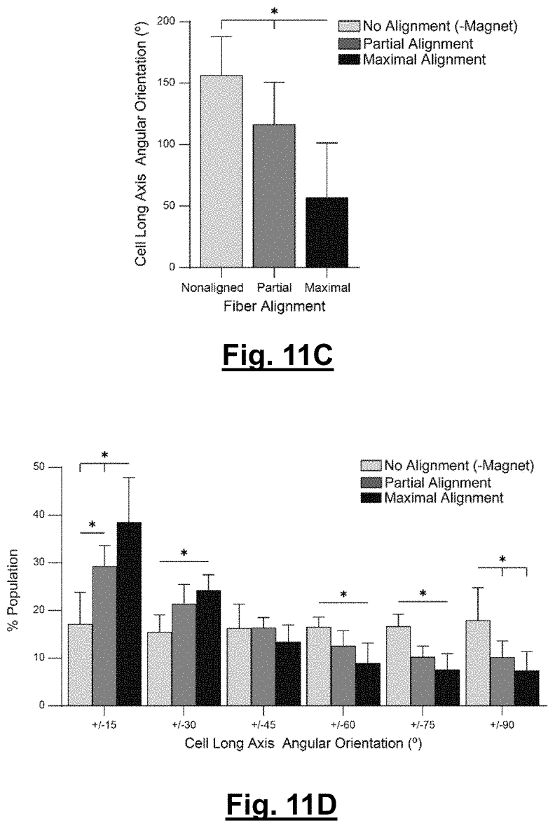

Alignment of fibrous ECM architecture is known to influence fibroblast spreading and polarization. For applications in tendon tissue engineering, alignment of tendon fibroblasts (tenocytes) within three-dimensional hydrogels may be critical to mechanosensing and ECM deposition. To enable fibroblast adhesion to magnetic fibers, residual VS groups are functionalized with the cell-adhesive peptide, CGRGDS (SEQ ID NO:2), via Michael-type addition. Primary tenocytes harvested from mouse tendons are coencapsulated along with SPION-containing fibers in a bulk MMP-degradable DVS hydrogel to determine the influence of fiber alignment on tenocyte spreading and orientation. Magnet spacing is modulated to produce aligned (6 cm), partially-aligned (12 cm), or nonaligned (no magnetic field) hydrogel composites. Tenocyte spreading in nonaligned gels includes both stellate morphologies in which filopodia extend in all directions (FIG. 11A, grey arrowheads) and uniaxial spread morphologies with high aspect ratios (FIG. 11A, white arrowheads). In contrast, tenocyte spreading in both partially-aligned and aligned gels favors higher aspect ratios with the long axes of cells oriented in the direction of fiber alignment. As shown in FIGS. 11B-11C, fiber alignment at the highest possible field strength (6 cm magnet spacing) results in significantly more aligned cells than lower field strength (12 cm magnet spacing), as calculated by the full width half max of cell orientation distributions. Quantification of individual cell orientations reveals nearly random distributions in nonaligned gels. In partially-aligned and aligned gels, the distribution of cell orientation increasingly narrows, with the majority of cells oriented within +/−30° of the fiber alignment axis, as shown in FIG. 11D.

Fiber Alignment Directs Multicellular Migration and Induces Migration Phenotype Switching.

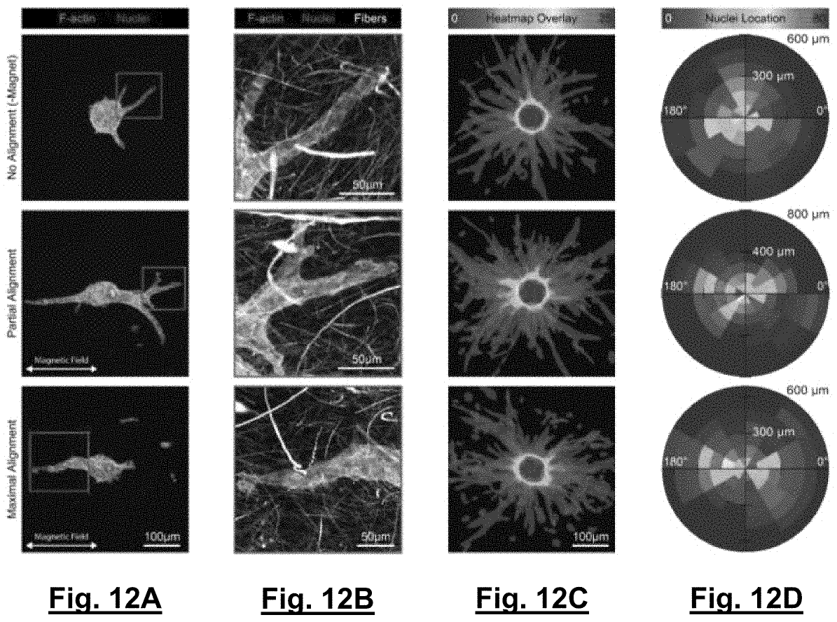

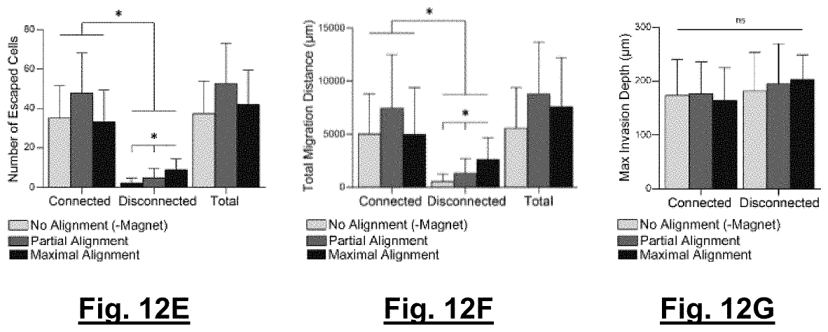

ECM fiber alignment has also been heavily implicated in epithelial cell migration during transtromal escape from primary tumors. To examine the effect of fiber alignment on epithelial cell migration, MCF10A spheroids and SPION-containing fibers are coencapsulated within MMP-degradable DVS gels. Degree of fiber alignment is again modulated by magnetic field strength. As shown in FIG. 12A and FIG. 13, migration from spheroids occurs predominantly as multicellular collective strands that contact guided along fiber segments bias in the direction of fiber alignment. Within partially-aligned and aligned fibrous matrices, nuclei also appear elongated in the direction of fiber alignment, as shown in FIG. 12B. To more directly visualize migration directional bias, a previously developed custom MATLAB image analysis code is utilized to generate heatmap overlays of actin structures (FIG. 12C) and rose plots of nuclear locations (FIG. 12D) for 25 spheroids. Nonaligned gels promote radially uniform cell outgrowths and distribution of nuclei. In contrast, the majority of migratory outgrowths in partially-aligned and maximally-aligned gels occurred within +/−30° of the axis of fiber alignment. A change in the total number of migrating cells across each gel condition is not observed, suggesting that fiber alignment does not increase the frequency of cell migration. However, in aligned gels, there was an increase in the number of cells migrating as single cells or multicellular clusters disconnected from the main body of the spheroid, as shown in FIG. 12E. The image analysis code also quantifies total migration distance (the summed migration distance of each cell from the spheroid periphery as a measure of net transtromal migration) and maximum invasion depth (the maximal depth into the surrounding stromal matrix of an outgrowth). Collective strands contiguous to the spheroid account for the majority of total transtromal migration distance, with no significant change as a function of fiber alignment. However, a significant increase in total migration distance of disconnected migratory cells is noted at both levels of fiber alignment, as shown in FIG. 12F. Despite the emergence of distinct migratory phenotypes, no change in maximum invasion distance is observed across different degrees of fiber alignment or between connected or disconnected phenotypes, as shown in FIG. 12G. In sum, these data suggest fiber alignment does not increase overall cell migration or migration speed, but rather increases directional migration via contact guidance and the frequency of cell-cell breakage events that engender disconnected invasive cell structures.

### Discussion

Here, a means to align magnetic electrospun fibers within a three-dimensional hydrogel composite that models stromal ECM is described. SPIONs are stably incorporated into DVS fiber segments, enabling control over the density and alignment of fibrous architecture via an externally applied magnetic field. SPION density and magnetic field strength jointly contribute to fiber alignment, enabling fine control over fiber alignment. Fiber entanglement due to the length and density of fiber segments impairs alignment, but shortening fibers via photomasking prevents fiber entanglement during magnetic alignment, thereby increasing the range of achievable fiber densities in three-dimensional hydrogel composites. Both the spreading of individually encapsulated cells and orientation of multicellular migratory structures from spheroids are influenced by the degree of fiber alignment. Aligned fibrous architecture directs uniaxial spreading of primary-derived mouse tenocytes in lieu of stellate morphologies. Fiber alignment also biases the direction of multicellular migration from MCF10A spheroids and increases the number of cell-cell breakage events, leading to the emergence of invading single cells and multicellular clusters. While previous methods have been developed to aligned fibers within purified biopolymer hydrogel, such as type I collagen, the synthetic fiber-reinforced hydrogel composite system presented provides more facile orthogonal tuning of fibrous architecture parameters, including the degree of alignment, fiber length, and fiber density.

The custom-designed magnetic gelation chamber holds two small N52 neodymium magnets that produce a surface field of 661.9 mT. In comparison to previous methods utilizing Tesla-range magnetic fields to align type I collagen gels, alignment of SPION-containing DVS fiber segments requires a significantly lower magnetic field strength achievable with small rare earth magnets without the need for specialized or expensive equipment (see FIGS. 6A-6C). In comparison to other methods of aligning fibers, such as flow-induced alignment or fibroblast-mediated matrix reorganization, control over magnetic fiber fabrication and field strength provide a higher degree of control of fiber alignment. As anticipated, fiber alignment id sensitive to both SPION density within fiber segments and field strength, as shown in FIGS. 7A-7D. As such, the degree of fiber alignment is tunable within hydrogel composites to produce different degrees of alignment. Enhanced control over the degree of alignment enables modeling of progressive stages of tissue repair or pathogenesis that involve matrix fibers, such as tendon regeneration or invasive ductal carcinomas, respectively. Furthermore, the degree of fiber alignment can be tuned to reflect histologic samples or in situ images of tissue to more accurately model specific tissue types or states of disease.

As the stroma possesses a high density of fibrous ECM proteins, fiber density within the hydrogel composites was modulated and fiber entanglement and reduced alignment is observed when the density of fibers exceeds 3 v/v %. To prevent entanglement, chrome photomasks were utilized to shorten fibers, as shown in FIGS. 9A-9G. Photomasking decreases variance in fiber lengths and enables increased alignment at higher fiber densities. The large variance in fiber length without photomasking is likely due to the processing of deposited fibers mats into individual fiber segments, which involves vortexing resuspended fiber mats—an uncontrolled process yielding fibers between 100-550 μm in length. In contrast, photomasking produces more consistent fiber lengths. Alignment of shorter fiber segments did not result in entanglement and proved insensitive to boundary effects, as shown in FIG. 9G. Fibers near the glass coverslip bottom and sides of the PDMS gasket are aligned to the same degree as fibers within the center of the gel. In comparison to flow-induced fiber alignment, which creates alignment artifacts near rigid boundaries, magnetic alignment readily overcomes initial fiber orientation resulting from the injection of hydrogel precursor solution.

Functionalization of fiber segments with cell-adhesive RGD allows cells to engage and spread along fiber segments and respond to matrix alignment. Following the encapsulation of primary-derived mouse tenocytes into aligned hydrogel composites, cell spreading along fiber segments and a morphologic transition from stellate to uniaxial morphologies oriented in the direction of fiber alignment is observed, as shown in FIGS. 11A-11D. Alignment of tenocytes has potential implications in tendon wound repair, as the cells and matrix within this tissue are highly organized. Given the non-contact nature of magnetic alignment, hydrogel composite precursor solutions containing tenocytes can be injected into the tendon wound site and then aligned, for example, with the use of a MRI machine or any other clinically available magnetic field generator.

Similar to single tenocyte spreading, multicellular migration from MCF10A spheroids biased in the direction of fiber alignment is observed, as shown in FIGS. 12A-12G. Cells migrating as collective strands are contact-guided along fiber segments, with nuclei elongated in the direction of fiber alignment. Interestingly, a significant increase in disconnected migratory outgrowths, including single cell and multicellular clusters with fiber alignment, is noted. This switch in migratory phenotype indicates that directional migration along aligned matrix fibers promotes EMT signaling and/or decreases cell-cell adhesion to induce cell-cell breakage events. Another explanation is that aligned matrices increase migration speed, causing leading cells to lose adhesion to slower moving trailing cells. Enhanced cell migration speed along aligned fibrous matrices has been reported in two-dimensional settings. While an increase in net invasion depth with fiber alignment is not observed, instantaneous migration speeds are not assessed here. As the bulk DVS hydrogel stiffness is separately defined from fiber density and alignment, this hydrogel composite can be used to investigate the individual contributions of fiber alignment and hydrogel stiffness on three-dimensional cell migration speed in future studies. Further investigation with timelapse imaging can directly assess if fiber alignment increases migration speed during proteolysis-dependent three-dimensional cell migration. Orthogonal tuning of fiber density and alignment at a constant hydrogel stiffness can also provide insight into the influence of tumor-associated collagen signatures (TACS), as previously described. TACS describes three major changes in collagen architecture surrounding solid tumors during breast cancer progression that facilitate metastatic invasion, two of which are increased fiber density and radial alignment of fibers at the tumor-stroma interface. By varying input volume fraction of fiber segments and magnetic field strength, matrix fiber density and alignment can be differentially tuned to model progressive states of tumor stroma.

While RGD is used to enable cell adhesion to fiber segments here, other ECM peptides can be used to model full length proteins, such as the GXOGER sequence (SEQ ID NO:3) of type I collagen. As DVS fiber segments are not hydrolytically or proteolytically degradable, they can also be used to study and model cell force-mediated reorganization of fibrous architecture. The magnetic electrospun fiber segments developed here can be easily integrated within other natural and synthetic biomaterials. An MMP-cleavable DVS hydrogel is selected as the bulk material here due to its tunability of bulk stiffness and crosslinking via Michael-type addition. For integration with other hydrogels, crosslinking kinetics should be carefully taken into account. Fiber segments are immobilized after 8 minutes of DVS hydrogel crosslinking via Michael-type addition. The post-gelation degree of fiber alignment is likely a function of magnetic field strength in conjunction with hydrogel precursor solution viscosity as a function of crosslinking. As such, stronger magnets may be required to achieve the same degree of fiber alignment if hydrogel crosslinking kinetics are significantly faster than the DVS hydrogels employed here.

A hydrogel composite system including SPION-containing electrospun fiber segments which can be aligned within an externally applied magnetic field is presented. Orthogonal tunability is demonstrated for key fibrous matrix attributes, including fiber length, fiber density, and degree of fiber alignment. The ability to align magnetic fibers proves insensitive to boundary conditions, allowing homogeneous fiber alignment throughout a millimeter-scale hydrogel. With this system, the ability to align single encapsulated primary mouse tenocytes is demonstrated, which may have utility as an injectable biomaterial therapy to mediate tendon repair. Furthermore, control over directional multicellular migration from MCF10A spheroids is shown, and it is found that fiber alignment induces breakage events, leading to migration phenotype switching from collective strands to single cells and multicellular clusters. The tunability of fibrous architecture within this hydrogel composite and the ability to integrate magnetic fibers with other biomaterials enables modeling of stromal tissue architectures in connective tissue repair and disease processes.

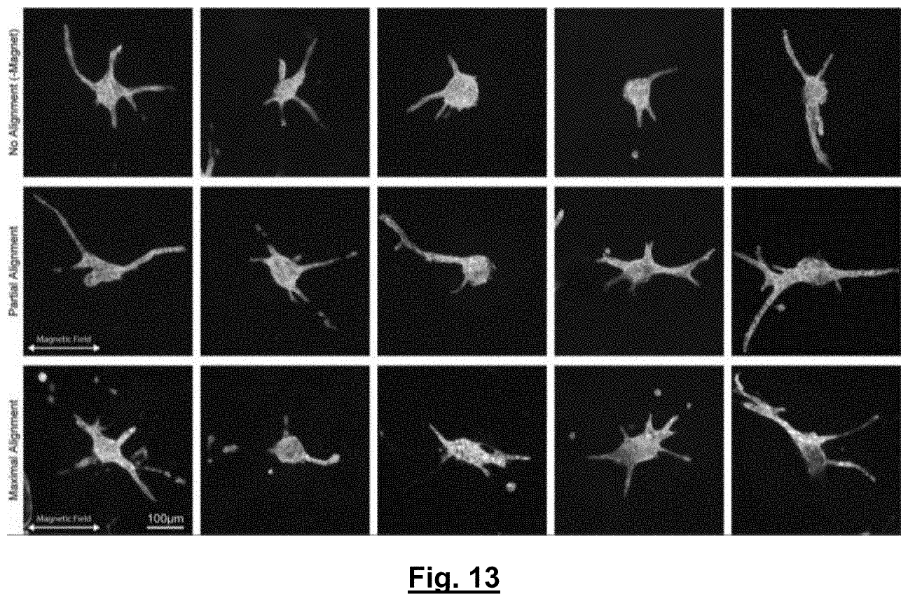

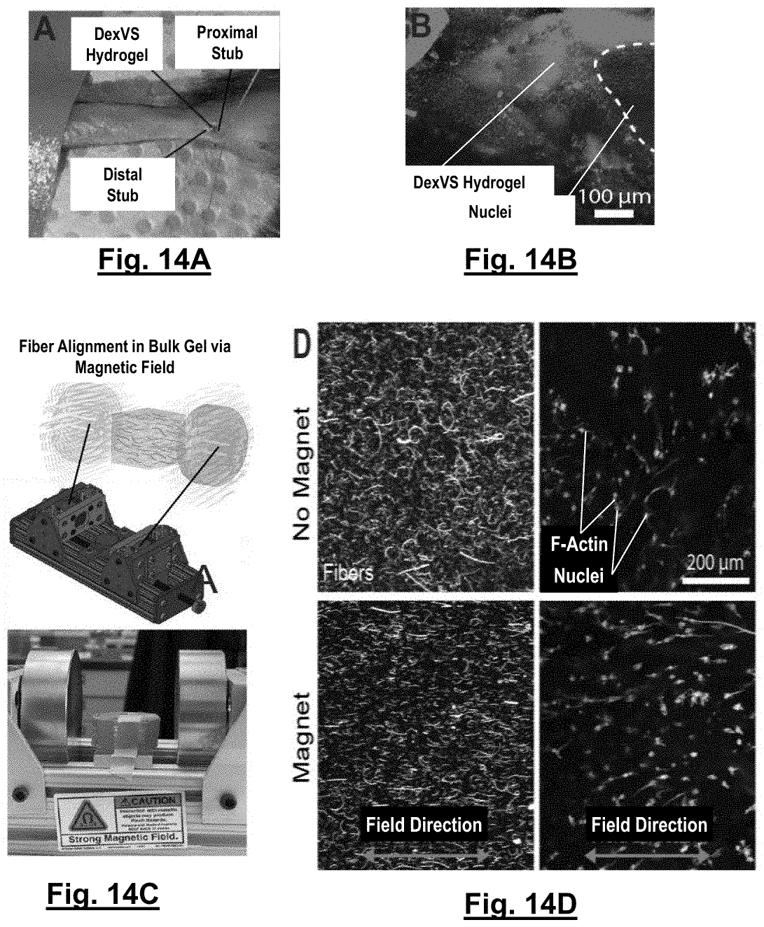

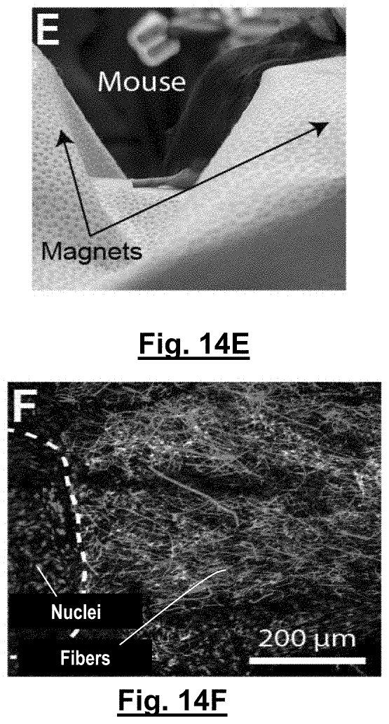

FIGS. 14A-14F show in vivo formation of a composite material comprising SPION-laden vinyl sulfone functionalized dextran (DexVS) fibers within hydrogels in a mouse in accordance with various aspects of the current technology. All animal studies were performed using 9-12 week old C57/B6 mice in accordance with IACUC animal care and NIH guidelines. Following anesthetization via isoflurane inhalation, a sharp tenotomy (full transection) of the Achilles tendon was performed. FIG. 14A shows intraoperative image from a tenotomy and hydrogel composite material implantation surgery.

The animal was transferred to the magnetic field device, positioning the hind limb in axis with the field direction. A composite hydrogel precursor solution containing magnetic fibers (SPION-laden vinyl sulfone functionalized dextran (DexVS) fibers) was prepared and injected into the gap spanning tendon stubs. Hydrogel gelation proceeded in the presence of the magnetic field for 10 minutes with a moistened segment of gauze placed above the wound site to prevent dehydration. FIG. 14C shows a schematic and image of a magnetic device for aligning SPION-laden DexVS fibers within hydrogels, which has a similar design to previous devices described above. FIG. 14E shows an image of isoflurane-sedated mouse with hind limb positioned between device magnets during tenotomy and gel implantation in accordance with certain aspects of the current technology. Hindlimbs were immobilized for the first week postoperatively to help stabilize the wound gap.

FIG. 14B shows a tendon/hydrogel construct composite material explanted 7 days post-operation. FIG. 14D shows confocal images of SPION fibers (left) and tendon progenitor cells (TPCs) (right) within composite hydrogels either gelled normally (top) or within the magnetic field device (bottom). FIG. 14F shows a confocal image of resulting fibrous hydrogel composite localized to the wound gap with fibers aligned along the long axis of the transected tendon.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A composite material comprising:
a hydrogel matrix having a three-dimensional geometry; and
fibers embedded and substantially uniformly distributed within the hydrogel matrix,
wherein the fibers have a substantially circular cross-sectional geometry, and
wherein the fibers are anisotropically aligned.

2. The composite material according to claim 1, wherein the hydrogel matrix comprises:
(i) a polysaccharide selected from the group consisting of dextran, starch, cellulose, alginate, hyaluronic acid, chitosan, chitin, pectin, derivatives thereof, and combinations thereof;
(ii) a polypeptide selected from the group consisting of collagen, fibronectin, gelatin, derivatives thereof, and combinations thereof;
(iii) a synthetic polymer comprising polyethylene glycol (PEG); or
(iv) any combination of (i)-(iii).

3. The composite material according to claim 1, wherein the fibers comprise:
(i) a polysaccharide selected from the group consisting of dextran, starch, cellulose, alginate, hyaluronic acid, chitosan, pectin, chitin, derivatives thereof, and combinations thereof;
(ii) a synthetic polymer selected from the group consisting of polyvinyl alcohol (PVA), polyethylene oxide (PEO), poly(hydroxyethyl methacrylate) (PHEMA), polyvinylpyrrolidone (PVP), polyimide (PI), polyacrylate (PA), polyurethane (PU), a polyester, and combinations thereof; or
(iii) a combination of (i) and (ii).

4. The composite material according to claim 1, wherein the fibers comprise a vinyl sulfone functionalized dextran.

5. The composite material according to claim 1, further comprising magnetic nanoparticles at least partially embedded within the fibers.

6. The composite material according to claim 4, wherein the magnetic nanoparticles are coated with a biomaterial.

7. The composite material according to claim 1, wherein the fibers are embedded within the hydrogel matrix at a fiber density of greater than or equal to about 1 v/v % to less than or equal to about 6 v/v %.

8. The composite material according to claim 1, wherein the hydrogel matrix is crosslinked with a peptide crosslinker.

9. The composite material according to claim 1, wherein the fibers are crosslinked with a peptide crosslinker.

10. The composite material according to claim 1, wherein cell adhesion promoters are coupled to at least one of the hydrogel matrix or the fibers.

11. The composite material according to claim 1, further comprising cells embedded within the hydrogel matrix.

12. The composite material according to claim 1, wherein the fibers have a fiber length of greater than or equal to about 100 micrometers to less than or equal to about 150 micrometers.

13. A method of producing a composite material, the method comprising:
preparing a suspension of electrospun fibers having a substantially circular cross-sectional geometry, where at least a portion of the electrospun fibers has at least one magnetic nanoparticle at least partially embedded therein;
combining the suspension with a hydrogel precursor solution comprising polymer molecules to form a composite suspension; and
crosslinking the polymer molecules within a magnetic field to form the composite material,
wherein the composite material comprises the electrospun fibers embedded and substantially uniformly distributed within a three-dimensional hydrogel matrix formed from the polymer molecules, and
wherein the electrospun fibers are anisotropically aligned.

14. The method according to claim 13, further comprising preparing the electrospun fibers by:
electrospinning a fiber mat comprising magnetic nanoparticles embedded within a plurality of continuous fibers;
disposing a photomask over the fiber mat, the photomask comprising a plurality of apertures;
applying ultraviolet (UV) light through the plurality of apertures to crosslink the continuous fibers at regions exposed beneath the apertures and to form the electrospun fibers;
isolating the electrospun fibers from portions of the fiber mat that were blocked from being crosslinked by the photomask; and
suspending the electrospun fibers in a solvent.

15. The method according to claim 14, wherein the apertures of the photomask have a diameter of greater than or equal to about 75 micrometers to less than or equal to about 250 micrometers and the electrospun fibers have a fiber length of greater than or equal to about 100 micrometers to less than or equal to about 150 micrometers.

16. The method according to claim 14, wherein the electrospinning is performed with a composition comprising a fiber precursor, a photoinitiator, and the magnetic nanoparticles at a density of greater than or equal to about 2.5 mg/mL to less than or equal to about 10 mg/mL.

17. The method according to claim 13, wherein the composite suspension is formed in, or transferred to, a sealed container, and the method further comprises periodically rotating the water-tight container about 180° to provide a substantially uniform distribution of the electrospun fibers within the composite suspension until the crosslinking is complete.

18. The method according to claim 13, further comprising generating the magnetic field between two magnets.

19. The method according to claim 13, wherein the magnetic field is characterized by a magnet flux density of greater than or equal to about 5 mT to less than or equal to about 1 T.

20. The method according to claim 13, further comprising adding a plurality of cells to the composite suspension.
